(19) United States
(12) Patent Application Publication
(10) Pub. No.: US 2018/0296066 A1
(43) Pub. Date: Oct. 18, 2018

(54) MEDICAL CLIP AND TREATMENT TOOL SYSTEM

(71) Applicant: OLYMPUS CORPORATION, Tokyo (JP)

(72) Inventors: Hitoshi KINOSHITA, Tokyo (JP); Atsushi BAN, Tokyo (JP)

(73) Assignee: OLYMPUS CORPORATION, Tokyo (JP)

(21) Appl. No.: 16/012,137

(22) Filed: Jun. 19, 2018

Related U.S. Application Data
(63) Continuation of application No. PCT/JP2016/086195, filed on Dec. 6, 2016.

(30) Foreign Application Priority Data
Dec. 21, 2015 (JP) 2015-248984

(51) Int. Cl. A61B 1/00; A61B 10/04

(52) U.S. Cl. CPC A61B 1/0014; A61B 1/267; A61B 1/00087; A61B 10/04

(57) ABSTRACT

A medical clip includes: a pair of knob sections constituted by a first knob and a second knob, the first knob and the second knob being formed so as to be separated from each other, the first knob being disposed parallel to a first portion having a flat plate shape, the second knob being disposed parallel to a second portion having a flat plate shape; a connecting section that connects the first portion and the second portion via a first bent section and a second bent section, wherein the first knob is connected to the first portion via a third bent section and the second knob is connected to the second portion via a fourth bent section; and an elastic member installed between the front end portion of the first knob and the front end portion of the second knob and elastically deformable.

### CROSS REFERENCE TO RELATED APPLICATIONS

This application is a continuation application based on a PCT Patent Application No. PCT/JP2016/086195, filed on Dec. 6, 2016, whose priority is claimed on Japanese Patent Application No. 2015-248984, filed on Dec. 21, 2015, the entire contents of which are hereby incorporated by reference.

### BACKGROUND

### Technical Field

The present invention relates to a medical clip and a treatment tool system.

### Background Art

In the related art, for example, when a biopsy is endoscopically performed on a peripheral lesion in a lung area, a target area is disposed in a bronchiole. In this case, a technique is known in which a guide sheath having a smaller diameter than an endoscope is inserted into a channel of the endoscope, the guide sheath extends from a tip of the endoscope and is placed in the vicinity of the target area and a treatment tool is inserted into the guide sheath to perform the biopsy. In this technique, the treatment tool inserted into the guide sheath may be exchanged with another treatment tool. When an insertion amount with respect to the guide sheath upon exchange of the treatment tool is inaccurate, a tip of the exchanged treatment tool may be separated from the target area. In response to the problem, a marker capable of accurately moving a tip of a treatment tool to a target area by being attached to the treatment tool is known (see Japanese Patent Publication No. 5019975).

The marker disclosed in Japanese Patent Publication No. 5019975 includes a main body having a substantially cylindrical shape and a fitting section installed on a front end portion of the main body, and an insertion hole through which a treatment tool as a mounting object is inserted is formed along a central axis of the marker. The marker is fixed to an arbitrary position of an intermediate portion of the treatment tool by a frictional force generated due to a contact of a tip portion of the insertion hole on the side of the fitting section with an outer surface of the treatment tool.

When the marker disclosed in Japanese Patent Publication No. 5019975 is attached to the treatment tool, the tip of the treatment tool is inserted through the insertion hole of the marker, and the marker is slid to a predetermined position on the treatment tool against a frictional force between the marker and the treatment tool. In many cases, a position to which the marker is fixed is separated about 1000 mm from the tip of the treatment tool. In this case, a marker sliding distance is long, and a force of a certain level should be applied to the marker when the marker is slid against the frictional force.

### SUMMARY

A medical clip attached to a treatment tool inserted and used in an endoscope, includes: a pair of knob sections formed to extend from front end portions to rear end portions and constituted by a first knob and a second knob, the first knob and the second knob being formed so as to be separated from each other, the first knob being disposed parallel to a first portion having a flat plate shape, the second knob being disposed parallel to a second portion having a flat plate shape; a connecting section including the first portion, the second portion, a first bent section, a second bent section, a third bent section, and a fourth bent section, wherein the first portion is connected to the second portion via the first bent section and the second bent section, the first knob is connected to the first portion via the third bent section, and the second knob is connected to the second portion via the fourth bent section; and an elastic member installed between the front end portion of the first knob and the front end portion of the second knob and elastically deformable. A through-hole is formed in the elastic member to pass through the elastic member in a direction crossing an opening and closing direction of the first knob and the second knob, and the elastic member has a slit formed to extend from the through-hole to an outer circumferential surface of the elastic member toward the front end portions of the pair of knob sections and having a gap in a state in which the front end portion of the first knob and the front end portion of the second knob approach and separate from each other.

The elastic member may further include an edge portion that forms a dihedral angle between an inner surface of the through-hole and an inner surface of the slit.

The medical clip may further include: a restricting member installed between the pair of knob sections and configured to restrict a movement range in which the front end portions of the pair of knob sections approach and separate from each other.

A fitting groove may be formed in communication with the through-hole and the slit and is formed in a surface of the elastic member crossing the direction about the through-hole. The fitting groove may be configured to be able to be fitted onto a base end portion of the guide sheath in a state in which the treatment tool is inserted into a guide sheath. The fitting groove may have an inner diameter smaller than an outer diameter of the base end portion of the guide sheath and larger than the inner diameter of the through-hole and is formed so as to be recessed from a surface of the elastic member crossing the direction.

A treatment tool system includes: a guide sheath formed to extend from a tip to a base end and able to be endoscopically inserted into a living body; a treatment tool having an insertion section that is able to be inserted into the guide sheath and a treatment section installed on a tip of the insertion section and configured to perform observation or treatment of the living body; and a medical clip detachably attached to the insertion section. The medical clip includes: a pair of knob sections formed to extend from front end portions to rear end portions and constituted by a first knob and a second knob, the first knob and the second knob being formed so as to be separated from each other, the first knob being disposed parallel to a first portion having a flat plate shape, the second knob being disposed parallel to a second portion having a flat plate shape; a connecting section including the first portion, the second portion, a first bent section, a second bent section, a third bent section, and a fourth bent section, wherein the first portion is connected to the second portion via the first bent section and the second bent section, the first knob is connected to the first portion via the third bent section, and the second knob is connected to the second portion via the fourth bent section; and an elastic member installed between the front end portion of the first knob and the front end portion of the second knob and elastically deformable. A through-hole is formed in the elastic member to pass through the elastic member in a direction crossing an opening and closing direction of the first knob and the second knob, and the elastic member has a slit formed to extend from the through-hole to an outer circumferential surface of the elastic member toward the front end portions of the pair of knob sections and having a gap in a state in which the front end portion of the first knob and the front end portion of the second knob approach and separate from each other.

The elastic member may further include an edge portion that forms a dihedral angle between an inner surface of the through-hole and an inner surface of the slit.

The medical clip may further include a restricting member installed between the pair of knob sections and configured to restrict a movement range in which the front end portions of the pair of knob sections approach and separate from each other.

The treatment tool may be any one of an ultrasound probe configured to observe a target tissue in the living body through ultrasound, forceps configured to grasp the target tissue, a brush configured to exfoliate a surface layer of the target tissue, and a guiding device configured to guide the guide sheath in the living body.

A method of applying a medical clip, includes: (1) inserting an ultrasound probe inserted through a guide sheath into a bronchiole; (2) causing an ultrasound probe to extend from a tip of the guide sheath and specifying a target area; (3) marking a mark on a base end of the ultrasound probe and defining a length of the ultrasound probe inserted into the guide sheath after defining the target area; (4) inserting and detaching the ultrasound probe into/from the guide sheath; (5) positioning the mark at the same position as a length of the ultrasound probe from the position at which the mark of the base end of the ultrasound probe is marked to a tip of the ultrasound probe by pinching a clip at a base end position of an insertion section of a treatment section from an outer side surface of the insertion section of the treatment section toward a major axis of the insertion section; (6) inserting the treatment section through the guide sheath and inserting a tip of the treatment section toward a target area, and causing the clip to butt against a base end surface of the guide sheath; and (7) treating a living body tissue in the target area using the treatment section in a state in which the clip butts against a base end of the guide sheath.

The clip may include a pair of knob sections formed to extend from front end portions to rear end portions and constituted by a first knob and a second knob, the first knob and the second knob being formed so as to be separated from each other. The pair of knob sections may be disposed parallel to a first portion having a flat pate shape and a second portion having a flat plate shape. The first portion and the second portion may have a connecting section connected by a first bent section and a second bent section. The pair of knob sections may be connected by a third bent section and a fourth bent section to connect the first portion and the second portion. An elastic member installed between the front end portion of the first knob and the front end portion of the second knob and elastically deformable may be provided. A through-hole may be formed in the elastic member to pass through the elastic member in a direction crossing a plane passing through the front end portion and the rear end portion of the pair of knob sections. The elastic member may have a slit having a gap in communication with the through-hole throughout the length of the through-hole and extending to an outer circumferential surface of the elastic member in a state in which the front end portion of the first knob and the front end portion of the second knob approach each other. Through the clip, the insertion section of the treatment section may be inserted from the slit toward the through-hole and further positioned.

The elastic member may include an edge portion that forms a dihedral angle between an inner surface of the through-hole and an inner surface of the slit, and the clip may be movable in a lengthwise direction of the insertion section of the treatment section and able to be detachably positioned from the insertion section by the edge portion.

A movement range in which the front end portions of the pair of knob sections approach and separate from each other may be restricted when a user opens and closes the pair of knob sections using a restricting member installed between the pair of knob sections and installed at rear end sides of the pair of knob sections.

### BRIEF DESCRIPTION OF THE DRAWINGS

FIG. 1 is a perspective view showing a medical clip according to a first embodiment of the present invention.

FIG. 2 is a view showing a frame of the medical clip.

FIG. 3A is a front view showing the medical clip.

FIG. 3B is an enlarged view of a portion L in FIG. 3A.

FIG. 4 is a right side view showing the medical clip.

FIG. 5 is a cross-sectional view taken along line A-A in FIG. 4.

FIG. 6 is a view showing a partial cross section of a guide sheath used with the medical clip.

FIG. 7 is a view showing an ultrasound probe serving as an example of a treatment tool used with the medical clip and the guide sheath.

FIG. 8 is a view showing forceps serving as another example of the treatment tool.

FIG. 9 is a view showing a brush serving as still another example of the treatment tool.

FIG. 10 is a view showing a guiding device serving as yet another example of the treatment tool.

FIG. 11 is a view showing a configuration of an endoscope used with a treatment tool system according to the first embodiment of the present invention.

FIG. 12 is a flowchart showing a sequence when a lung biopsy is performed by the treatment tool using the treatment tool system.

FIG. 13 is a view showing a state in which the medical clip is attached to the forceps.

FIG. 14 is a cross-sectional view taken along line B-B in FIG. 13.

FIG. 15 is a view showing a state in which the endoscope approaches the vicinity of a target area.

FIG. 16 is a view showing a state in which the ultrasound probe extends from the endoscope.

FIG. 17 is a view showing a state in which an image of the target area is acquired by the ultrasound probe.

FIG. 18 is a view showing a state in which the guide sheath is placed in the target area.

FIG. 19 is a view showing a state in which the medical clip attached to the forceps abuts the guide sheath.

FIG. 20 is a view showing a state in which a tissue of the target area is collected using the forceps.

FIG. 21 is a flowchart showing another sequence when a lung biopsy is performed by the treatment tool using the treatment tool system.

FIG. 22 is a view showing a state in which an image of the target area is acquired by the ultrasound probe.

FIG. 23 is a view showing a use example of the medical clip.

FIG. 24 is a view showing a variant of the medical clip.

FIG. 25 is a perspective view showing a medical clip according to a second embodiment of the present invention.

FIG. 26 is a front view showing the medical clip.

FIG. 27 is a cross-sectional view showing a cross section corresponding to a cross section of the medical clip shown in FIG. 5 in the medical clip.

FIG. 28 is a front view showing a medical clip according to a third embodiment of the present invention.

FIG. 29 is a cross-sectional view taken along line C-C in FIG. 28.

FIG. 30 is a view showing a state in which the medical clip attached to forceps is fitted to a base end portion of a guide sheath.

FIG. 31 is a view showing a medical clip according to a fourth embodiment of the present invention.

FIG. 32 is a view showing a medical clip according to a fifth embodiment of the present invention.

FIG. 33 is a view showing a use example of the medical clip.

### DETAILED DESCRIPTION OF THE PREFERRED EMBODIMENTS

### First Embodiment

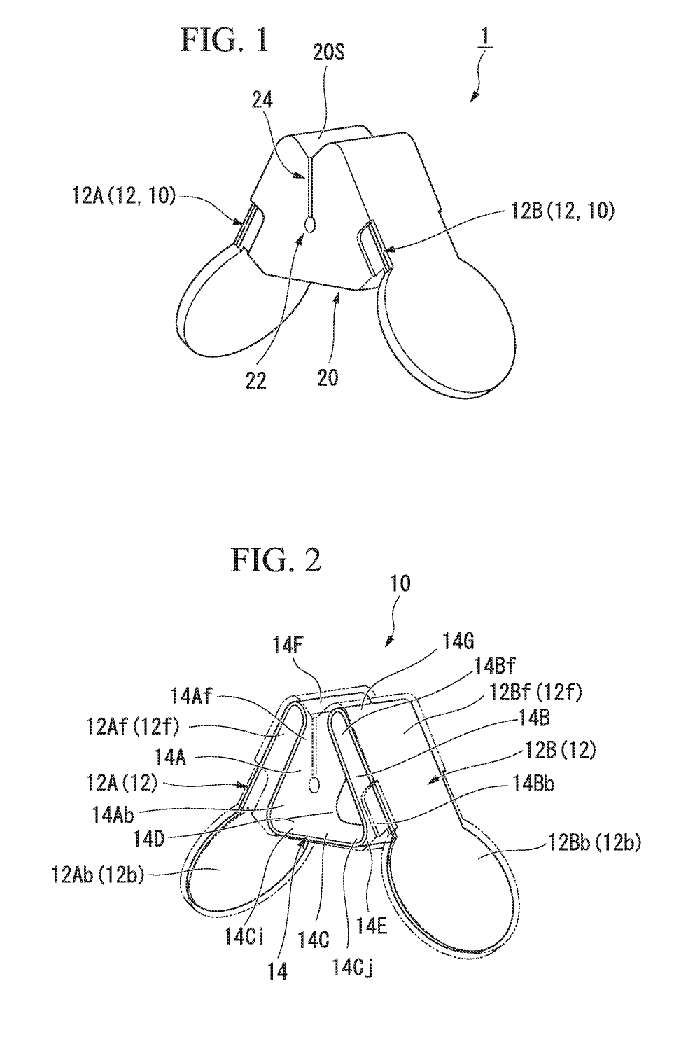

Hereinafter, a first embodiment of the present invention will be described with reference to FIGS. 1 to 24.

FIG. 1 is a perspective view showing a medical clip 1 according to the first embodiment. The medical clip 1 includes a frame 10 and an elastic member 20.

FIG. 2 is a view showing the frame 10 of the medical clip 1. In FIG. 2, a two-dotted chain line shows an appearance of the medical clip 1. The frame 10 includes a pair of knob sections 12 constituted by a first knob 12A and a second knob 12B, and a connecting section 14 configured to connect the pair of knob sections 12, i.e., the first knob 12A and the second knob 12B. In addition, in the embodiment, the frame 10 is formed by folding a plate-shaped member having rigidity.

The pair of knob sections 12 are formed to extend from front end portions 12f to rear end portions 12b and to be separated from each other. In the embodiment, in the pair of knob sections 12, the first knob 12A has a front end portion 12Af (121) and a rear end portion 12Ab (12b), and is formed to extend from the front end portion 12Af to the rear end portion 12Ab. Similarly, the second knob 12B has a front end portion 12Bf (12f) and a rear end portion 12Bb (12b), and is formed to extend from the front end portion 12Bf to the rear end portion 12Bb. The first knob 12A and the second knob 12B are formed such that the front end portion 12Af and the front end portion 12Bf face each other and the rear end portion 12Ab and the rear end portion 12Bb face each other.

In addition, in the embodiment, the pair of knob sections 12 are formed so as to be separated from each other from the front end portions 12f toward the rear end portions 12b. Specifically, the first knob 12A and the second knob 12B are formed so as to be separated from each other from the front end portion 12Af and the front end portion 12Bf toward the rear end portion 12Ab and the rear end portion 12Bb. That is, the first knob 12A and the second knob 12B are disposed such that a distance between the rear end portion 12Ab and the rear end portion 12Bb is larger than a distance between the front end portion 12Af and the front end portion 12Bf.

The rear end portion 12Ab of the first knob 12A and the rear end portion 12Bb of the second knob 12B are portions held by a user via the elastic member 20 installed on surfaces of the rear end portion 12Ab and the rear end portion 12Bb when used. In the embodiment, each of the rear end portion 12Ab and the rear end portion 12Bb is formed in a disk shape.

The connecting section 14 connects the pair of knob sections 12 such that the front end portions 12f of the pair of knob sections 12 can approach or separate from each other. In the embodiment, the connecting section 14 is disposed between the pair of knob sections 12 and constituted by a first portion 14A having a flat plate shape, a second portion 14B having a flat plate shape, a third portion 14C having a flat plate shape, a first bent section 14D configured to connect the first portion 14A and the third portion 14C, a second bent section 14E configured to connect the second portion 14B and the third portion 14C, a third bent section 14F configured to connect the first knob 12A and the first portion 14A, and a fourth bent section 14G configured to connect the second knob 12B and the second portion 14B.

The first portion 14A is disposed substantially parallel to the first knob 12A. The first portion 14A has a front end portion 14Af connected to the front end portion 12Af of the first knob 12A via the third bent section 14F, and a rear end portion 14Ab connected to the first bent section 14D. The second portion 14B is disposed substantially parallel to the second knob 12B. The second portion 14B has a front end portion 14Bf connected to the front end portion 12Bf of the second knob 12B via the fourth bent section 14G, and a rear end portion 14Bb connected to the second bent section 14E. The third portion 14C has a first end portion 14Ci connected to the first bent section 14D, and a second end portion 14Cj connected to the second bent section 14E. The first bent section 14D connects the first portion 14A and the third portion 14C in a state in which the first portion 14A is inclined with respect to the third portion 14C such that the front end portion 14Af of the first portion 14A approaches the second end portion 14Cj of the third portion 14C. The second bent section 14E connects the second portion 14B and the third portion 14C in a state in which the second portion 14B is inclined with respect to the third portion 14C such that the front end portion 14Bf of the second portion 14B approaches the first end portion 14Ci of the third portion 14C. That is, the connecting section 14 is formed in a substantially triangular shape when seen in a cross section parallel to a plate thickness direction.

While the frame 10 is constituted by a member having rigidity, since the connecting section 14 has the above-mentioned configuration, the frame 10 can be elastically deformed within a predetermined range by applying a force to cause the rear end portions 12b of the pair of knob sections 12 to approach each other. For example, when a force is applied such that the rear end portions 12b of the pair of knob sections 12 approach each other, the first bent section 14D is deformed such that the first portion 14A is separated from the third portion 14C, and the second bent section 14E is deformed such that the second portion 14B is separated from the third portion 14C. Accordingly, the front end portion 12Af of the first knob 12A and the front end portion 12Bf of the second knob 12B are separated from each other while the front end portion 14Af of the first portion 14A and the front end portion 14Bf of the second portion 14B are separated from each other. That is, the front end portions 12f of the pair of knob sections 12 are separated from each other. In addition, when a force applied to the rear end portions 12b of the pair of knob sections 12 is removed, the first bent section 14D and the second bent section 14E that are deformed are returned to their original shapes. Accordingly, the front end portions 12f of the pair of knob sections 12 are returned to their original states from the state in which the front end portions 12f are separated from each other. That is, the front end portions 12f of the pair of knob sections 12 approach each other.

Accordingly, in the embodiment, the pair of knob sections 12 are connected by the connecting section 14 having the above-mentioned configuration such that the front end portions 12f can approach and separate from each other.

When using the medical clip 1, as will be described below, the treatment tool is disposed between the pair of knob sections 12. For this reason, in the frame 10, the first knob 12A and the second knob 12B are disposed to be separated from each other such that the treatment tool can be disposed between the first knob 12A and the second knob 12B. In addition, a dimension of the frame 10 in the widthwise direction (a direction perpendicular to a plane including a plate thickness direction) is set to a dimension that enables the medical clip 1 to reliably hold the treatment tool.

A metal material that is a member having rigidity may be used as a material of the frame 10. For example, stainless steel or the like is exemplified as the metal material. In addition, in the embodiment, while the frame 10 is formed by folding a plate-shaped or beltlike member, there is no limitation thereto and the frame 10 may be constituted by a plurality of members.

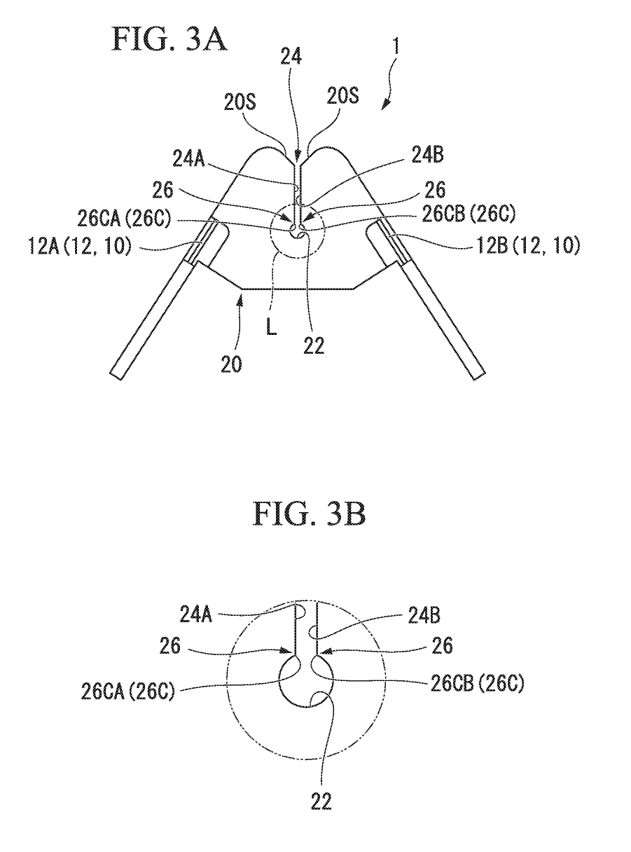

FIG. 3A is a front view showing the medical clip 1. FIG. 3B is an enlarged view of a portion L in FIG. 3A. The elastic member 20 is elastically deformable and installed between the front end portions 12f of the pair of knob sections 12. Further, in the embodiment, in consideration of the ease of fabricating the medical clip 1, while the elastic member 20 is installed to cover the entire frame 10, this is not an essential configuration. As shown in FIG. 3A, the elastic member 20 has a through-hole 22 formed to pass through the elastic member 20, and a slit 24 formed to come in communication with the through-hole 22 throughout the length of the through-hole 22 and extending to an outer circumferential surface 20S of the elastic member 20.

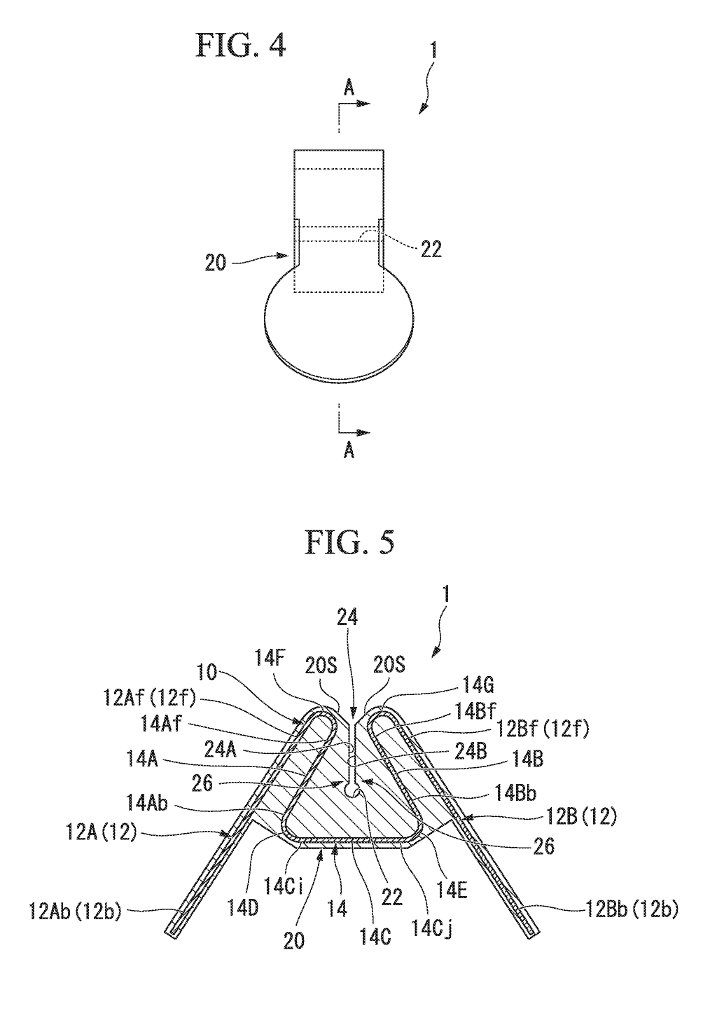

FIG. 4 is a right side view showing the medical clip 1. FIG. 5 is a cross-sectional view taken along line A-A in FIG. 4. The through-hole 22 is formed to pass through the elastic member 20 in a direction (hereinafter, referred to as a first direction) crossing a plane passing through the front end portions 12f and the rear end portions 12b of the pair of knob sections 12.

Specifically, in the embodiment, a plane (hereinafter, referred to as a first plane) shown by line A-A in FIG. 4 is employed as shown in FIG. 5 as a plane passing through the front end portions 12f and the rear end portions 12b of the pair of knob sections 12. Also, a direction (hereinafter, referred to as a second direction) perpendicular to the first plane as the first direction is employed. Accordingly, in the embodiment, the through-hole 22 passes through the elastic member 20 in the second direction. In addition, the through-hole 22 is formed in the elastic member 20 installed between the front end portions 12f of the pair of knob sections 12. Accordingly, the through-hole 22 is disposed between the front end portions 12f of the pair of knob sections 12.

The slit 24 is configured to be opened and closed according to approaching or separation of the front end portions 12f of the pair of knob sections 12. In the embodiment, since the through-hole 22 is formed in the elastic member 20 installed between the front end portions 12f of the pair of knob sections 12, the through-hole 22 is disposed between the front end portions 12f of the pair of knob sections 12. Here, the elastic member 20 installed between the front end portions 12f of the pair of knob sections 12 is fixed to the front end portions 12f, i.e., the front end portion 12Af of the first knob 12A and the front end portion 12Bf of the second knob 12B. Alternatively, the elastic member 20 is connected to so as to move according to approaching or separation of the front end portions 12f. In addition, the slit 24 is formed to extend from the through-hole 22 to the outer circumferential surface 20S on the front end side of the elastic member 20 in a direction in which the front end portions 12f approach each other between the front end portions 12f of the pair of knob sections 12. That is, the slit 24 passes through the elastic member 20 from the through-hole 22 to the outer circumferential surface 20S. Simultaneously, the slit 24 passes through the elastic member 20 in the second direction.

Since the slit 24 has the above-mentioned configuration, the slit 24 can be opened and closed according to approaching or separation of the front end portions 12f of the pair of knob sections 12. For example, when the front end portions 12f of the pair of knob sections 12 are separated from each other, among inner surfaces that constitute the slit 24, a first inner surface 24A that is the inner surface disposed at a position close to the first knob 12A is moved together with the first knob 12A so as to be separated from the second knob 12B, and a second inner surface 24B that is the inner surface disposed at a position close to the second knob 12B is moved together with the second knob 12B so as to be separated from the first knob 12A. That is, since the first inner surface 24A and the second inner surface 24B are moved so as to be separated from each other, the slit 24 is opened. In addition, when the front end portions 12f of the pair of knob sections 12 approach each other from a state in which they are separated from each other, the first inner surface 24A and the second inner surface 24B approach each other from a state in which they are separated from each other. That is, the slit 24 is closed from an open state. In this way, in the embodiment, the slit 24 is configured to be opened and closed according to approaching and separation of the front end portions 12f of the pair of knob sections 12. In addition, since the elastic member 20 is configured to be elastically deformable, the elastic member 20 is appropriately elastically deformed according to opening and closing of the slit 24. That is, when the slit 24 is opened from a closed state, the elastic member 20 is elastically deformed as the inner diameter of the through-hole 22 is enlarged according to the operation. When the slit 24 is closed, the elastic member 20 is elastically deformed such that the inner diameter of the enlarged through-hole 22 is returned to its original dimension.

The medical clip 1 holds the treatment tool by fitting the insertion section of the treatment tool into the through-hole 22. Accordingly, since the insertion section of the treatment tool is reliably held in the through-hole 22, the through-hole 22 has an inner diameter smaller than an outer diameter of the insertion section of the treatment tool in a state in which the through-hole 22 is not elastically deformed. When the treatment tool is held in the through-hole 22, the elastic member 20 is elastically deformed such that the through-hole 22 follows the outline of the insertion section of the treatment tool. Accordingly, since the outer circumferential surface of the insertion section comes in surface contact with the inner surface of the through-hole 22 while the elastic member 20 presses the insertion section using a recovering force of returning the elastic member 20 to a shape before elastic deformation, the treatment tool can be more reliably held. In addition, a gap of the slit 24 in a state in which there is no elastic deformation, i.e., a distance between the first inner surface 24A and the second inner surface 24B is smaller than an inner diameter of the through-hole 22. Accordingly, the treatment tool held in the through-hole 22 cannot be easily removed from the slit 24.

In addition, as described above, since the elastic member 20 is elastically deformed such that the through-hole 22 follows the outline of the insertion section of the treatment tool when the medical clip 1 is attached to the treatment tool, the medical clip 1 having the same shape can be attached to the plurality of treatment tools having different outer diameters of the insertion sections.

In addition, the elastic member 20 further has an edge portion 26 installed between the through-hole 22 and the slit 24. As shown in FIG. 3B, the edge portion 26 forms a dihedral angle 26C between the inner surface of the through-hole 22 and the inner surface of the slit 24. In the embodiment, the dihedral angle 26CA is formed between the inner surface of the through-hole 22 and the first inner surface 24A of the slit 24, and the dihedral angle 26CB is formed between the inner surface of the through-hole 22 and the second inner surface 24B of the slit 24. Due to the edge portion 26 configured as described above, movement range of the treatment tool held in the through-hole 22 to the slit 24 is prevented by the dihedral angle 26C. For this reason, the treatment tool cannot be easily removed from the through-hole 22, and the treatment tool can be more reliably held.

In the embodiment, the elastic member 20 in which the through-hole 22 is formed is disposed to be surrounded by the frame 10 having rigidity when seen in the second direction. Specifically, the elastic member 20 in which the through-hole 22 is formed is surrounded and supported by the first portion 14A, the second portion 14B and the third portion 14C of the connecting section 14 when seen in the second direction. Since the elastic member 20 has lower rigidity than the frame 10, the treatment tool cannot be sufficiently held by only the elastic member 20. For this reason, since the elastic member 20 in which the through-hole 22 is formed is supported by the frame 10 (in the embodiment, the connecting section 14), rigidity of the entire configuration can be increased, and the treatment tool can be more reliably held. In this way, the frame 10 functions as a reinforcement member configured to reinforce the elastic member 20.

In the embodiment, the elastic member 20 has a dimension in the second direction (in the embodiment, coincides with a widthwise direction of the frame 10) substantially equal to a dimension of the frame 10 in the widthwise direction. However, the dimension of the elastic member 20 in the second direction is not particularly limited as long as a dimension enables the treatment tool to be held in the through-hole 22, and may be smaller or larger than the dimension of the frame 10 in the widthwise direction. In addition, the entire dimension of the elastic member 20 in the second direction is set to a dimension that can abut the base end portion of the guide sheath used with the medical clip 1. For example, the entire dimension of the elastic member 20 in the second direction is set to a dimension larger than an outer diameter of the base end portion of the guide sheath.

A member having elasticity may be used as a material of the elastic member 20. For example, a silicon rubber, a resin material having elasticity, or the like, is exemplified as such a member.

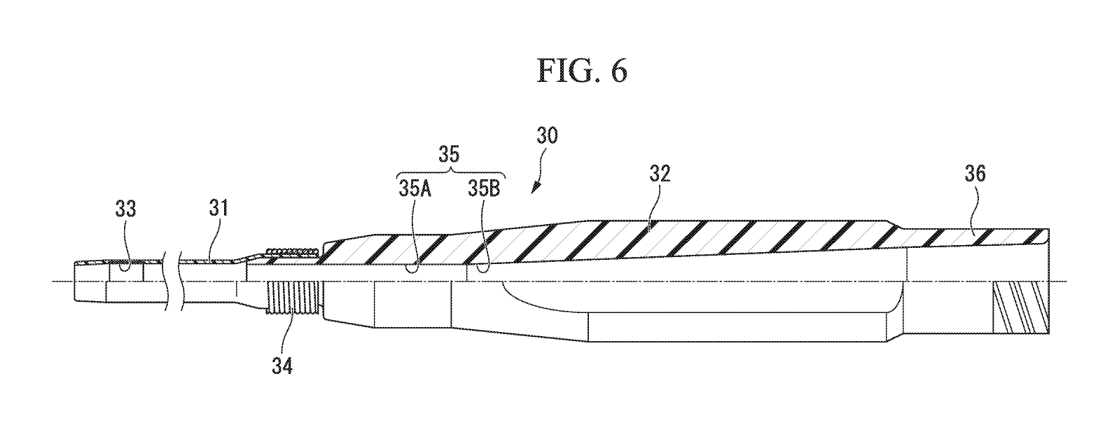

Next, a guide sheath 30 used with the medical clip 1 will be described. FIG. 6 is a view showing a partial cross section of the guide sheath 30. The guide sheath 30 is formed to extend from a tip to a base end, and can be endoscopically inserted into a living body. The guide sheath 30 includes a sheath main body 31 having a tubular shape, and a cock 32 attached to the base end of the sheath main body 31.

The sheath main body 31 is formed of a resin or the like and has flexibility. A tubular index member 33 is fixed to an inner surface of the sheath main body 31 close to a tip portion thereof by thermoforming. The index member 33 is formed of stainless steel or the like and is a reference for a tip position of the sheath main body 31 under X-ray fluoroscopy. A base end of the sheath main body 31 is press-fitted onto the tip of the cock 32 and fixed to the tip of the cock 32 by a coil 34 installed outside thereof.

The cock 32 is a tubular member formed of a resin or the like. The cock 32 has a through-hole 35 formed therein. The treatment tool can be inserted through the through-hole 35. The through-hole 35 has a circular cross section, and is constituted by a front half section 35A having a constant inner diameter, and a rear half section 35B having an inner diameter that is thickened toward a base end thereof. The inner surface of the rear half section 35B is formed in a tapered shape such that a wall surface of the cock 32 is increased from a base end portion 36 in the axial direction of the cock 32. When the medical clip 1 is used with the guide sheath 30, the medical clip 1 abuts the base end portion 36 of the guide sheath 30.

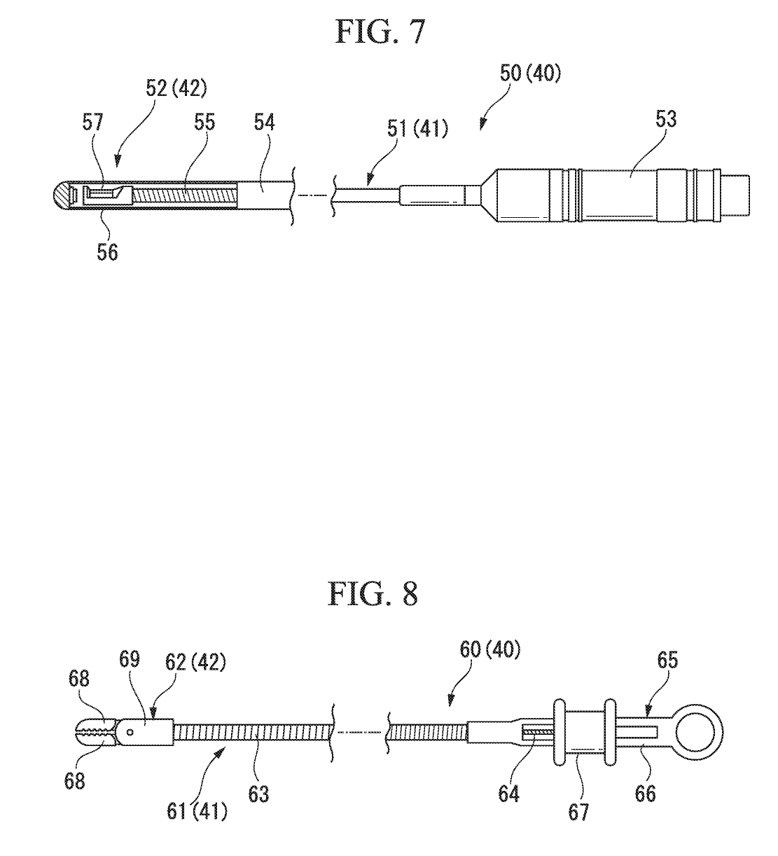

Next, a treatment tool 40 used with the medical clip 1 and the guide sheath 30 will be described. The treatment tool 40 has an insertion section 41 that can be inserted into the guide sheath 30, and a treatment section 42 installed on the tip of the insertion section 41 and configured to perform observation or treatment of a living body. FIGS. 7 to 10 show an example of the treatment tool 40.

FIG. 7 is a view showing an ultrasound probe 50 as an example of the treatment tool 40. The ultrasound probe 50 observes a target area (a target tissue) in the living body using ultrasound vibrations. The ultrasound probe 50 has an insertion section 51 that can be inserted into the guide sheath 30, and a treatment section 52 installed on the tip of the insertion section 51 and configured to perform observation of the living body. The insertion section 51 has flexibility and is formed in an elongated shape. A connector section 53 is formed on the base end of the insertion section 51. The connector section 53 is mechanically and electrically connected to a driving apparatus (not shown). In addition, the insertion section 51 has a sheath 54 having an outer diameter that can be inserted into the guide sheath 30, and a flexible shaft 55 rotatably inserted through the sheath 54. The flexible shaft 55 is inserted into a cover 56 of the treatment section 52. An ultrasound transducer 57 is attached to the tip portion of the flexible shaft 55 in the cover 56. The ultrasound transducer 57 is an element configured to receive a signal transmitted from a cable (not shown) through the flexible shaft 55 to oscillate ultrasound waves, and receive the ultrasound waves to convert the received ultrasound waves into an electrical signal when the ultrasound waves strike a test object and are returned. The cover 56 is filled with an ultrasound medium such that transmission of the ultrasound waves becomes easy.

FIG. 8 is a view showing forceps 60 as another example of the treatment tool 40. The forceps 60 grasp a target area (a target tissue). The forceps 60 have an insertion section 61 that can be inserted into the guide sheath 30, and a treatment section 62 installed on the tip of the insertion section 61 and configured to perform treatment of the living body. The insertion section 61 has flexibility and is formed in an elongated shape. The insertion section 61 has a sheath 63 having an outer diameter that can be inserted into the guide sheath 30, and an operating wire 64 inserted through the sheath 63. An operation section 65 is installed on the base end of the insertion section 61. The operation section 65 has an operation section main body 66 and a slider 67 attached to the operation section main body 66 so as to be capable of advancing and retreating. The treatment section 62 has a pair of grasping sections 68 and a support member 69 configured to support the pair of grasping sections 68 so as to open and close the grasping sections 68. The pair of grasping sections 68 are connected to one end of the operating wire 64 via a link mechanism (not shown). The other end of the operating wire 64 is connected to the slider 67. The pair of grasping sections 68 are opened when the operating wire 64 advances, and the pair of grasping sections 68 are closed when the operating wire 64 retreats.

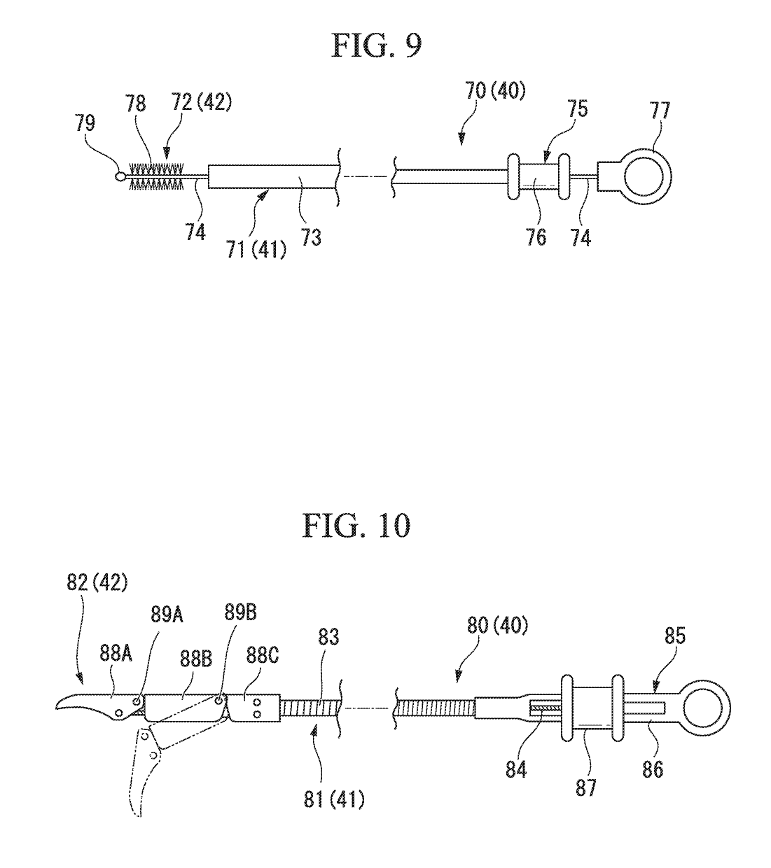

FIG. 9 is a view showing a brush 70 as still another example of the treatment tool 40. The brush 70 exfoliates a surface layer of the target area (the target tissue). The brush 70 has an insertion section 71 that can be inserted into the guide sheath 30, and a treatment section 72 installed on the tip of the insertion section 71 and configured to perform treatment of the living body. The insertion section 71 has flexibility and is formed in an elongated shape. The insertion section 71 has a sheath 73 having an outer diameter that can be inserted into the guide sheath 30, and an operating wire 74 inserted through the sheath 73. An operation section 75 is installed on the base end of the insertion section 71. The operation section 75 has an operation section main body 76, and a finger hook ring 77 installed on the base end side of the operation section main body 76. The operating wire 74 passes through the operation section main body 76 so as to be capable of advancing and retreating, and the finger hook ring 77 is fixed to the end portion drawn out from the operation section main body 76. The treatment section 72 has a brush section 78 attached to the tip portion of the operating wire 74. The brush section 78 can be accommodated in the sheath 73 of the insertion section 71. When the brush section 78 is accommodated in the sheath 73, a tip chip 79 formed on the tip of the operating wire 74 butts against the tip of the insertion section 71.

FIG. 10 is a view showing a guiding device 80 as yet another example of the treatment tool 40. The guiding device 80 guides the guide sheath 30 in the living body. In addition, the guiding device 80 can collect cells in the target area (the target tissue). The guiding device 80 has an insertion section 81 that can be inserted into the guide sheath 30, and a treatment section 82 installed on the tip of the insertion section 81 and configured to perform treatment of the living body. The insertion section 81 has flexibility and is formed in an elongated shape. The insertion section 81 has a sheath 83 having an outer diameter that can be inserted into the guide sheath 30, and an operating wire 84 inserted through the sheath 83. An operation section 85 is provided at the base end of the insertion section 81. The operation section 85 has an operation section main body 86, and a slider 87 attached to the operation section main body 86 so as to be capable of advancing and retreating. The treatment section 82 has a first arm section 88A, a second arm section 88B and a third arm section 88C. The first arm section 88A, the second arm section 88B and the third arm section 88C are disposed from the tip to the base end in sequence. A base end side of the first arm section 88A and a tip side of the second arm section 88B are connected by a first pin 89A. A base end side of the second arm section 88B and a tip side of the third arm section 88C are connected by a second pin 89B. A cup configured to collect a tissue is formed in the first arm section 88A. The operating wire 84 is inserted through the third arm section 88C, the second arm section 88B and the first arm section 88A and fixed to the first arm section 88A. Accordingly, as shown by a two-dotted chain line in FIG. 10, since the operating wire 84 is pulled toward the base end side, the treatment section 82 can be bent using the first pin 89A and the second pin 89B as fulcrums. In addition, when the operating wire 84 is returned to the tip side from a state in which the treatment section 82 is bent, the treatment section 82 extends linearly.

Further, in addition to the above examples of the above-mentioned treatment tool 40, another treatment tool such as a biopsy needle or the like may be used in combination with the medical clip 1 and the guide sheath 30.

A treatment tool system 90 according to the embodiment includes the medical clip 1, the guide sheath 30 and the treatment tool 40, which are described above. The medical clip 1 is detachably attached to the insertion section 41 of the treatment tool 40, and configured to abut the base end portion 36 of the guide sheath 30.

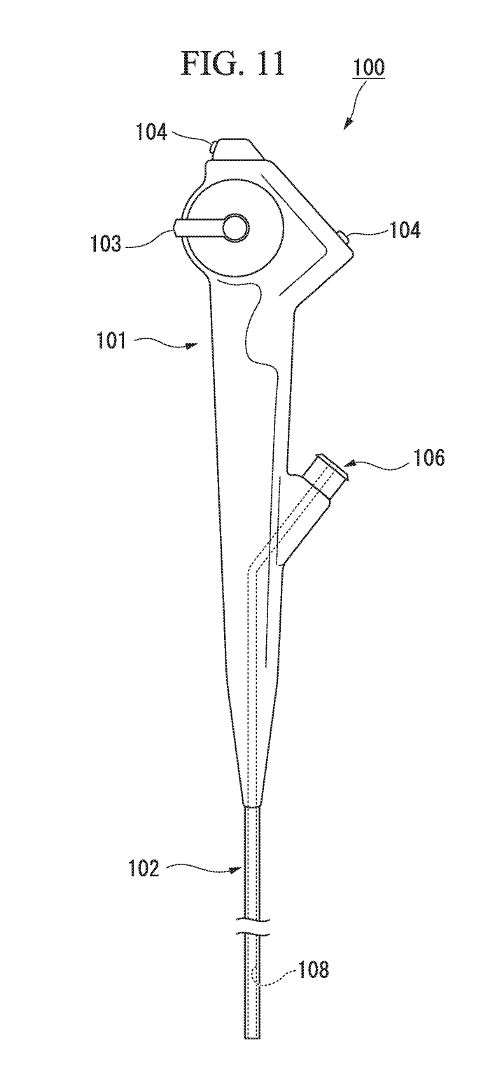

Next, an endoscope 100 used with the treatment tool system 90 will be described. FIG. 11 is a view showing a configuration of the endoscope 100. The endoscope 100 has an operation section 101 operated by a user, and an insertion section 102 formed continuous from the operation section 101. The insertion section 102 can be inserted into the body, has flexibility and is formed in an elongated shape. A channel 108 through which the guide sheath 30, the treatment tool 40, or the like, can be inserted is formed in the insertion section 102. An operating lever 103 configured to bend the tip portion of the insertion section 102 and a switch 104 are installed on the operation section 101. In addition, the operation section 101 is connected to a control device, a display device, or the like (not shown), via a universal cable (not shown). An insertion port 106 is formed on a side portion of the operation section 101. The insertion port 106 is an opening outside the body of the channel 108 and in communication with an opening of the tip of the insertion section 102 via the channel 108. Further, an endoscope having a known configuration may be used as the endoscope 100.

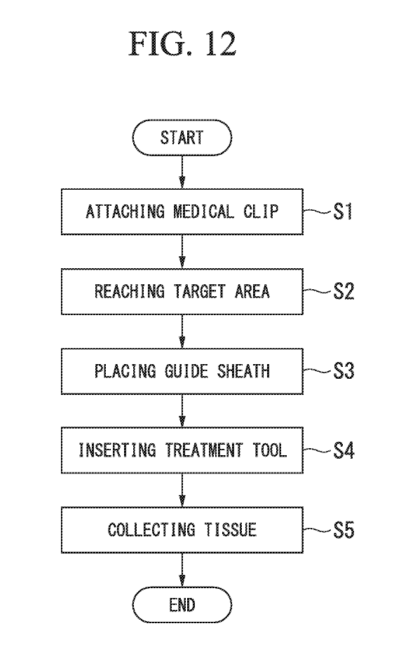

Next, an operation of the treatment tool system 90 during use will be described with reference to FIGS. 12 to 21 as an example of a lung biopsy technique. FIG. 12 is a flowchart showing a sequence when a lung biopsy is performed by the treatment tool 40 using the treatment tool system 90. A sequence of the flowchart shown in FIG. 12 will be described below as Procedure Example 1.

### Procedure Example 1

In step S1 shown in FIG. 12, the medical clip 1 is attached to the ultrasound probe 50 and the forceps 60 used as the treatment tool 40 in this procedure. First, a protrusion amount of the ultrasound probe 50 from the tip of the guide sheath 30 is previously set. The medical clip 1 is attached to the insertion section 51 at a position corresponding to the protrusion amount. Next, a protrusion amount of the forceps 60 from the tip of the guide sheath 30 is set. The protrusion amount of the forceps 60 is set such that a protrusion amount of the ultrasound probe 50 from the tip of the guide sheath 30 at a position of the ultrasound transducer 57 is equal to a protrusion amount of the forceps 60 from the tip of the guide sheath 30 at a position of the grasping sections 68. Another medical clip 1 is attached to the insertion section 61 at a position corresponding to the protrusion amount.

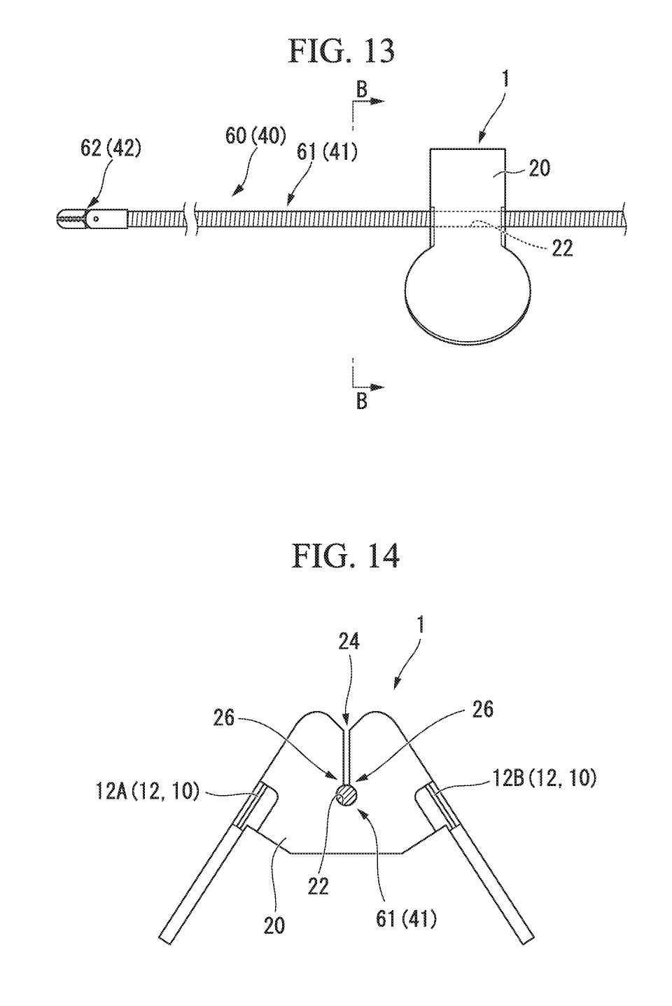

FIG. 13 is a view showing a state in which the medical clip 1 is attached to the forceps 60. As shown in FIG. 13, in a state in which the slit 24 of the medical clip 1 is open, the insertion section 61 (the insertion section 41) of the forceps 60 (the treatment tool 40) is inserted from the radial direction and the insertion section 61 is disposed in the through-hole 22 of the medical clip 1. As the slit 24 is closed from this state, the insertion section 61 is engaged with the through-hole 22 and the medical clip 1 is attached to the forceps 60. FIG. 14 is a cross-sectional view taken along line B-B in FIG. 13, and schematically showing the insertion section 61. As shown in FIG. 14, in a state in which the insertion section 61 is engaged with the through-hole 22, the elastic member 20 is elastically deformed such that the inner diameter of the through-hole 22 is increased following the outline of the insertion section 61. Accordingly, since the outer circumferential surface of the insertion section 61 comes in surface contact with the inner surface of the through-hole 22 while the elastic member 20 presses the insertion section 61 using a recovering force of returning the elastic member 20 to a shape before elastic deformation, the forceps 60 are more reliably held. In addition, positioning of the medical clip 1 is performed by moving the medical clip 1 to a position at which the protrusion amount of the forceps 60 is a set value in the forceps 60 while appropriately opening and closing the slit 24 of the medical clip 1. In the medical clip 1, since the inner diameter of the through-hole 22 is increased in a state in which the slit 24 is open, engagement between the insertion section 61 of the forceps 60 and the through-hole 22 is relaxed, and a frictional force generated between the outer circumferential surface of the insertion section 61 and the inner surface of the through-hole 22 when the medical clip 1 is moved along the insertion section 61 may be reduced or eliminated. For this reason, the medical clip 1 can be smoothly moved along the insertion section 61 and positioning of the medical clip 1 can be easily performed. Further, even for the ultrasound probe 50, the medical clip 1 is attached as described above.

Next, in step S2 shown in FIG. 12, the guide sheath 30 reaches a target area (a target tissue) R where the tissue is collected. Specifically, the endoscope 100 advances into the bronchus while recognizing a position of the target area R using an X-ray fluoroscopic image.

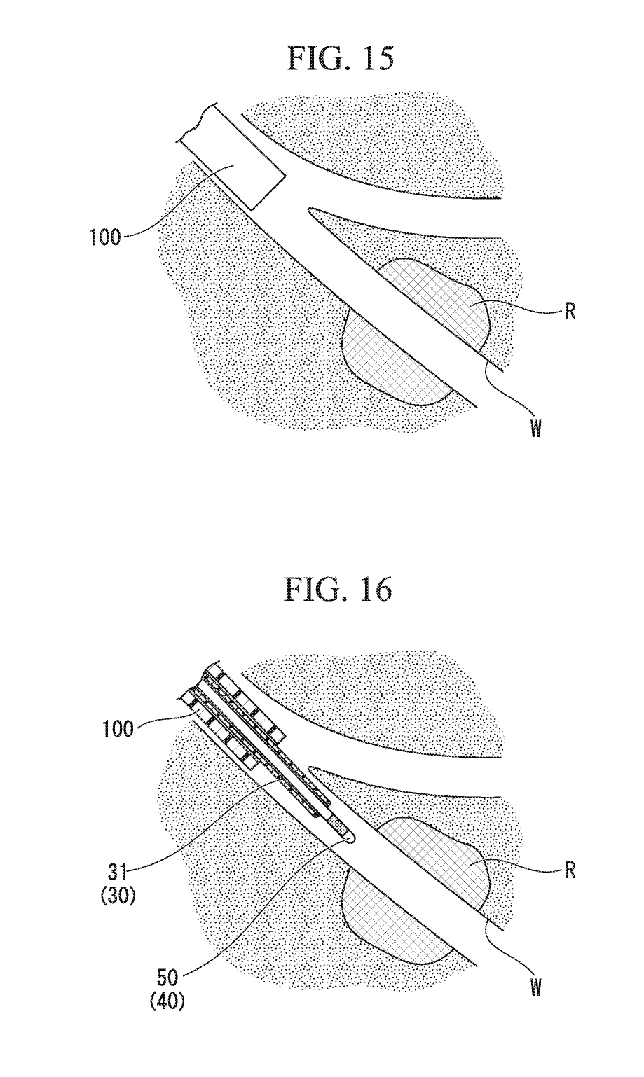

FIG. 15 is a view showing a state in which the endoscope 100 advances to the vicinity of the target area R. A diameter of a bronchiole W in which the target area R is present is smaller than a diameter of the endoscope 100. For this reason, the endoscope 100 cannot approach the target area R any more.

Here, the ultrasound probe 50 inserted through the guide sheath 30 is inserted from the insertion port 106 of the endoscope 100 together with the guide sheath 30. FIG. 16 is a view showing a state in which the ultrasound probe 50 extends from the endoscope 100. As shown in FIG. 16, the ultrasound probe 50 extends from the tip of the endoscope 100. Here, as the medical clip 1 abuts the base end portion 36 of the guide sheath 30, the ultrasound probe 50 extends from the tip of the guide sheath 30 by a set protrusion amount. Since the sheath main body 31 of the guide sheath 30 and the ultrasound probe 50 have a diameter smaller than the endoscope 100, they can reach the target area R through the bronchiole W.

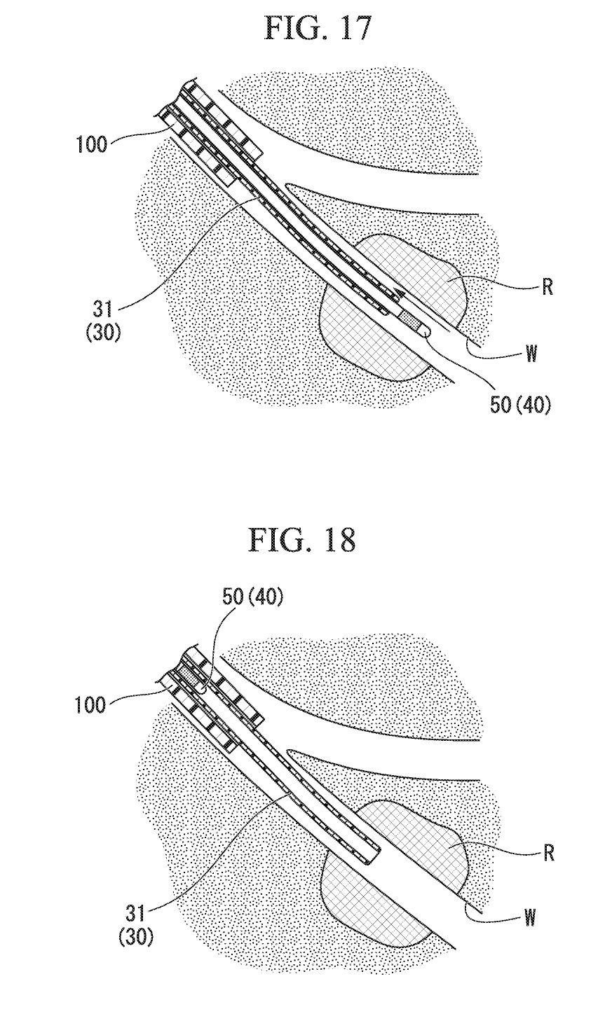

FIG. 17 is a view showing a state in which an image of the target area R is acquired by the ultrasound probe 50. In a state in which the medical clip 1 abuts the base end portion 36 of the guide sheath 30, as shown in FIG. 17, the ultrasound probe 50 advances to an area that seems to be a portion deeper than the target area R together with the sheath main body 31. Next, as shown by an arrow in FIG. 17, the ultrasound probe 50 is slowly pulled backward together with the sheath main body 31. Here, the tissue image around the ultrasound probe 50 obtained using the ultrasound waves emitted from the ultrasound transducer 57 is displayed on an inspection apparatus (not shown). Accordingly, it is possible to recognize whether the ultrasound probe 50 has reached the target area R.

If it is recognized that the ultrasound probe 50 has reached the target area R, the sequence advances to step S3. In a state in which the ultrasound probe 50 has reached the target area R, the position of the guide sheath 30 protruding from the insertion port 106 of the endoscope 100 is marked such that an insertion amount of the guide sheath 30 into the body can be known. For example, a known guide sheath stopper (not shown) may be used as a marker. In this case, the guide sheath stopper is previously attached to the sheath main body 31 of the guide sheath 30. Then, the guide sheath stopper is moved to a position at which the guide sheath stopper abuts the insertion port 106 in a state in which the ultrasound probe 50 has reached the target area R, and this becomes the mark of the insertion amount of the guide sheath 30.

FIG. 18 is a view showing a state in which the guide sheath 30 is placed in the target area R. As shown in FIG. 18, only the ultrasound probe 50 is drawn out from the guide sheath 30 while holding the guide sheath 30 at the position of the mark. Accordingly, the guide sheath 30 is placed in the vicinity of the target area R.

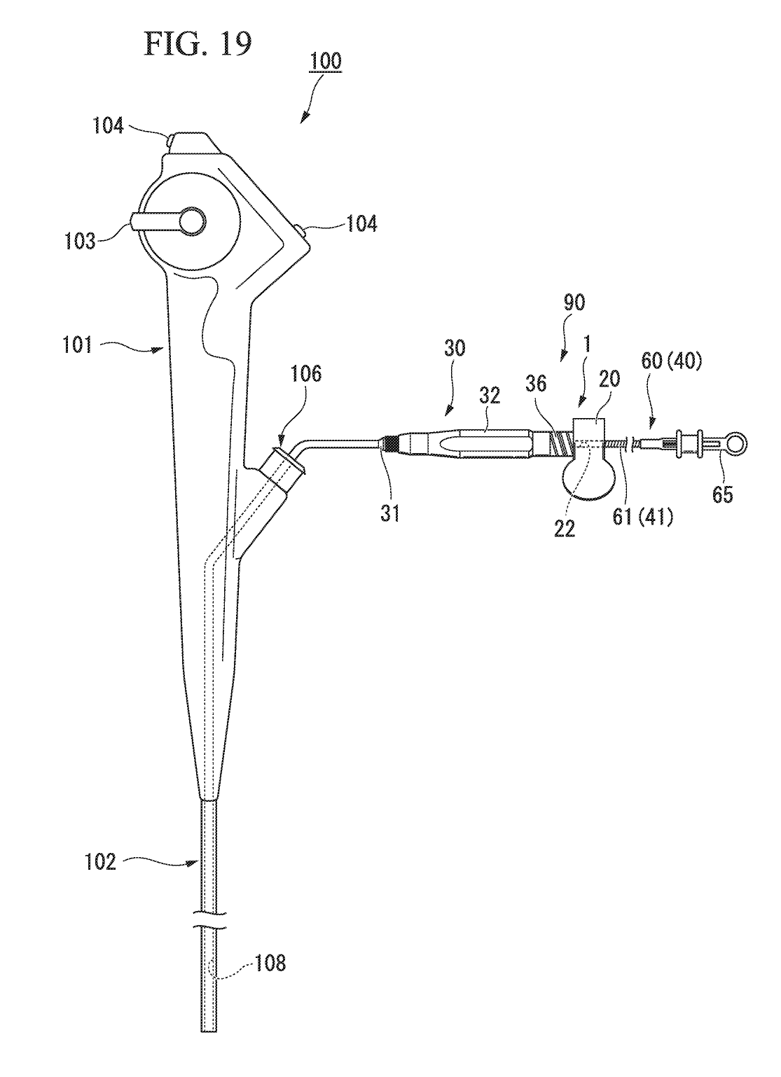

Next, the sequence advances to step S4. FIG. 19 is a view showing a state in which the medical clip 1 attached to the forceps 60 abuts the guide sheath 30. Further, in FIG. 19, illustration of the tip side of the insertion section 102 is omitted. In step S4, the forceps 60 to which the medical clip 1 is attached is inserted into the guide sheath 30 from the base end of the cock 32. Then, as shown in FIG. 19, the medical clip 1 abuts the base end portion 36 of the guide sheath 30.

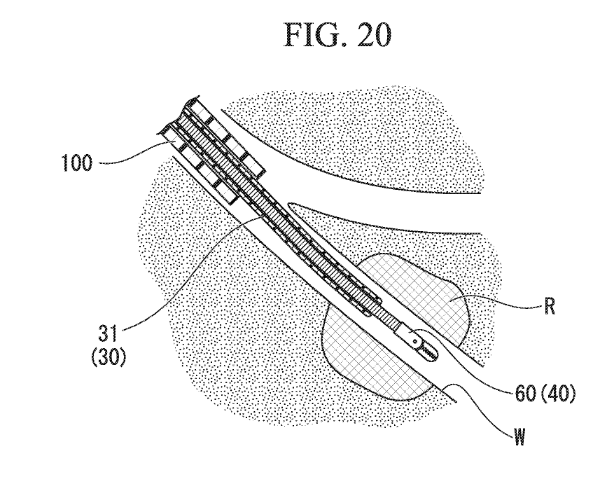

FIG. 20 is a view showing a state in which the tissue of the target area R is collected using the forceps 60. In a state in which the medical clip 1 abuts the base end portion 36 of the guide sheath 30, the forceps 60 extend from the tip of the sheath main body 31 by a protrusion amount set in step S1. For this reason, as shown in FIG. 20, the tip of the forceps 60 reliably reaches the target area R.

Next, in step S5, the forceps 60 are operated to collect the tissue of the target area R using the treatment section 62. After collecting of the tissue, the forceps 60 are withdrawn and the gathered tissue is collected. Tissue collecting in step S5 may be repeated several times according to necessity. In addition, a position at which tissue collecting is impossible can be specified by finely adjusting the position of the medical clip 1 and moving the tip position of the forceps 60 back and forth to repeat tissue collecting several times, and a size of the target area R (a length of the bronchiole W in a traveling direction) can also be estimated from the position.

### Procedure Example 2

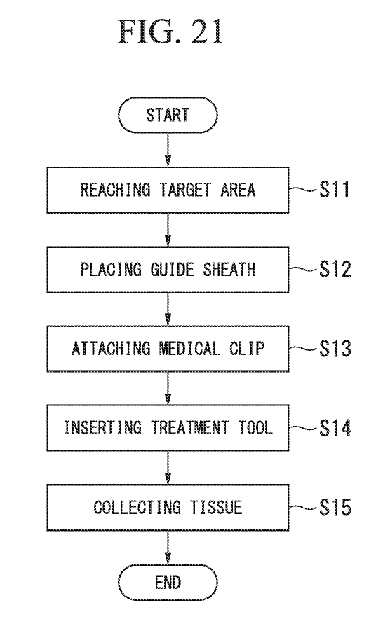

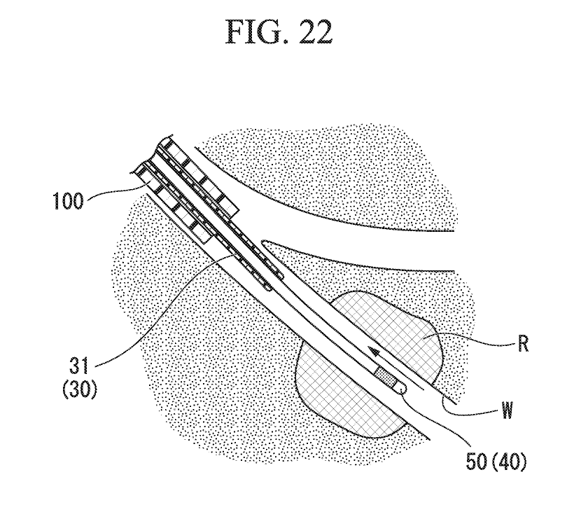

Instead of the above-mentioned Procedure Example 1, the technique may be performed by Procedure Example 2 as described below. FIG. 21 is a flowchart showing a sequence when a lung biopsy is performed by the treatment tool 40 using the treatment tool system 90 as Procedure Example 2. The flowchart of Procedure Example 2 shown in FIG. 21 is different from the flowchart of Procedure Example 1 shown in FIG. 12, and step S13 of attaching the medical clip is performed after step S12 of placing the guide sheath. Hereinafter, Procedure Example 2 will be described with reference to FIGS. 13 to 16 and FIGS. 18 to 20, which are used for description of Procedure Example 1, and FIG. 22.

First, in step S11 shown in FIG. 21, the guide sheath 30 reaches the target area R in which the tissue is collected. Specifically, the endoscope 100 advances into the bronchus while recognizing the position of the target area R using an X-ray fluoroscopic image.

As shown in FIG. 15, a diameter of the bronchiole W where the target area R is present is smaller than a diameter of the endoscope 100. For this reason, the endoscope 100 cannot approach the target area R any more.

Here, the ultrasound probe 50 (the treatment tool 40) inserted through the guide sheath 30 is inserted from the insertion port 106 of the endoscope 100 together with the guide sheath 30. As shown in FIG. 16, the ultrasound probe 50 extends from the tip of the endoscope 100. Since the sheath main body 31 of the guide sheath 30 and the ultrasound probe 50 have a diameter smaller than that of the endoscope 100, they can reach the target area R through the bronchiole W.

As shown in FIG. 22, the sheath main body 31 is stopped at a position that seems to be in front of the target area R, and the ultrasound probe 50 advances to an area that seems to be a portion deeper than the target area R. Next, as shown by an arrow in FIG. 22, the ultrasound probe 50 is slowly pulled backward. Here, the tissue image around the ultrasound probe 50 obtained using the ultrasound waves emitted from the ultrasound transducer 57 is displayed on an inspection apparatus (not shown). Accordingly, it is possible to recognize whether the ultrasound probe 50 has reached the target area R.

If it is recognized that the ultrasound probe 50 has reached the target area R, the sequence advances to step S12. In step S12, the sheath main body 31 advances to the vicinity of the target area R along the ultrasound probe 50. The tip position of the sheath main body 31 is confirmed by the index member 33 (not shown).

Here, in a state in which the tip of the ultrasound probe 50 is disposed at a position where tissue collecting is to be performed, the position of the ultrasound probe 50 protruding from the base end of the cock 32 of the guide sheath 30 is marked. Then, as shown in FIG. 18, only the ultrasound probe 50 is drawn out from the guide sheath 30, and the guide sheath 30 is placed in the vicinity of the target area R.

Next, the sequence advances to step S13. As shown in FIG. 13, the medical clip 1 is attached to the forceps 60. Here, the medical clip 1 is moved to a position at which a length from the position in the forceps 60 to which the medical clip 1 is attached to the tip of the forceps 60 is equal to a length from the tip of the ultrasound probe 50 to the mark marked in step S12 while appropriately opening and closing the slit 24 of the medical clip 1, and positioning of the medical clip 1 is performed. Further, since an operation of attaching the medical clip 1 to the forceps 60 is the same as in Procedure Example 1, description thereof will be omitted.

Next, the sequence advances to step S14. In step S14, the forceps 60 to which the medical clip 1 is attached is inserted into the guide sheath 30 from the base end of the cock 32. Then, as shown in FIG. 19, the medical clip 1 abuts the base end portion 36 of the guide sheath 30.

In a state in which the medical clip 1 abuts the base end portion 36 of the guide sheath 30, the forceps 60 extend from the tip of the sheath main body 31 by the same length as the ultrasound probe 50 in step S12. Then, as shown in FIG. 20, the tip of the forceps 60 reliably reaches the target area R.

Next, in step S15, the forceps 60 are operated to collect the tissue of the target area R using the treatment section 62. The operation in step S15 is the same as the operation in step S5 of Procedure Example 1.

Further, in the above-mentioned Procedure Example 1 and Procedure Example 2, instead of the forceps 60 used as the treatment tool 40, another treatment tool such as the brush 70, the guiding device 80, or the like, may be used.

In step S2 of Procedure Example 1 and step S11 of Procedure Example 2, instead of using the ultrasound probe 50, the forceps 60 passing through the guide sheath 30 may directly reach the target area R, or the guide sheath 30 may solely reach the target area R. In addition, the guide sheath 30 may be guided to the target area R using the guiding device 80. Guidance to the target area R may be performed by appropriately selecting X-ray fluoroscopy, an ultrasound wave image, an endoscope image, or the like.

In step S12 of Procedure Example 2, when the guide sheath 30 is placed in the vicinity of the target area R, a known guide sheath stopper (not shown) configured to determine a protrusion amount of the guide sheath 30 from the endoscope 100 may be used in the guide sheath 30. The guide sheath stopper can be attached to the sheath main body 31 of the guide sheath 30 and can fix the guide sheath 30 to the insertion port 106 of the endoscope 100.

According to the medical clip 1 and the treatment tool system 90 of the embodiment, the through-hole 22 of the elastic member 20 has an inner diameter smaller than the outer diameter of the insertion section 41 of the treatment tool 40. For this reason, in a state in which the insertion section 41 is engaged with the through-hole 22, the elastic member 20 is elastically deformed such that the through-hole 22 follows the outline of the insertion section 41. Accordingly, since the outer circumferential surface of the insertion section 41 comes in surface contact with the inner surface of the through-hole 22 while the elastic member 20 presses the insertion section 41 using a recovering force of the elastic member 20, the treatment tool 40 can be reliably held. In addition, the slit 24 of the elastic member 20 is formed to come in communication with the through-hole 22 throughout the length of the through-hole 22 and extend to the outer circumferential surface 20S of the elastic member 20, and configured to be opened and closed by approaching and separation of the front end portions 12f of the pair of knob sections 12. For this reason, the insertion section 41 can be easily disposed in the through-hole 22 through the slit 24 that can be opened and closed, and the insertion section 41 disposed in the through-hole 22 can be easily removed from the through-hole 22. Accordingly, attachment and removal of the medical clip 1 to/from the treatment tool 40 can be simply performed.

In addition, the edge portion 26 of the elastic member 20 forms the dihedral angle 26C between the inner surface of the through-hole 22 and the inner surface of the slit 24. For this reason, movement range of the treatment tool 40 held by the through-hole 22 to the slit 24 is prevented by the dihedral angle 26C. Accordingly, the treatment tool 40 cannot be easily removed from the through-hole 22, and the treatment tool 40 can be more reliably held.

Further, in the description of the above-mentioned embodiment, while the pair of knob sections 12 are formed so as to be separated from each other from the front end portions 12f toward the rear end portions 12b, the configuration is only exemplary. The pair of knob sections 12 may be provided apart from each other.

In addition, the configuration of the connecting section 14 in the description of the above-mentioned embodiment is only exemplary, and the configuration is not particularly limited as long as the pair of knob sections 12 are able to be connected to cause the front end portions 12f of the pair of knob sections 12 to approach and separate from each other.

In addition, in the above-mentioned description, while a first plane that is a plane shown by line A-A in FIG. 4 is employed as a plane passing through the front end portions 12f and the rear end portions 12b of the pair of knob sections 12, this is only exemplary. The plane is not limited to the first plane and may be a plane passing through the front end portions 12f and the rear end portions 12b. In addition, while a second direction perpendicular to the first plane is employed as the direction in which the through-hole 22 passes through the elastic member 20, the second direction is only exemplary and may be a direction crossing the first plane.

In the description of the above-mentioned embodiment, for example, as shown in FIG. 3A, while the slit 24 is configured such that a gap is formed between the first inner surface 24A and the second inner surface 24B, the configuration is only exemplary. The slit 24 may be formed such that the first inner surface 24A and the second inner surface 24B come in contact with each other. That is, the slit 24 includes a configuration in which the first inner surface 24A and the second inner surface 24B are separated from each other and a configuration in which the first inner surface 24A and the second inner surface 24B come in contact with each other.

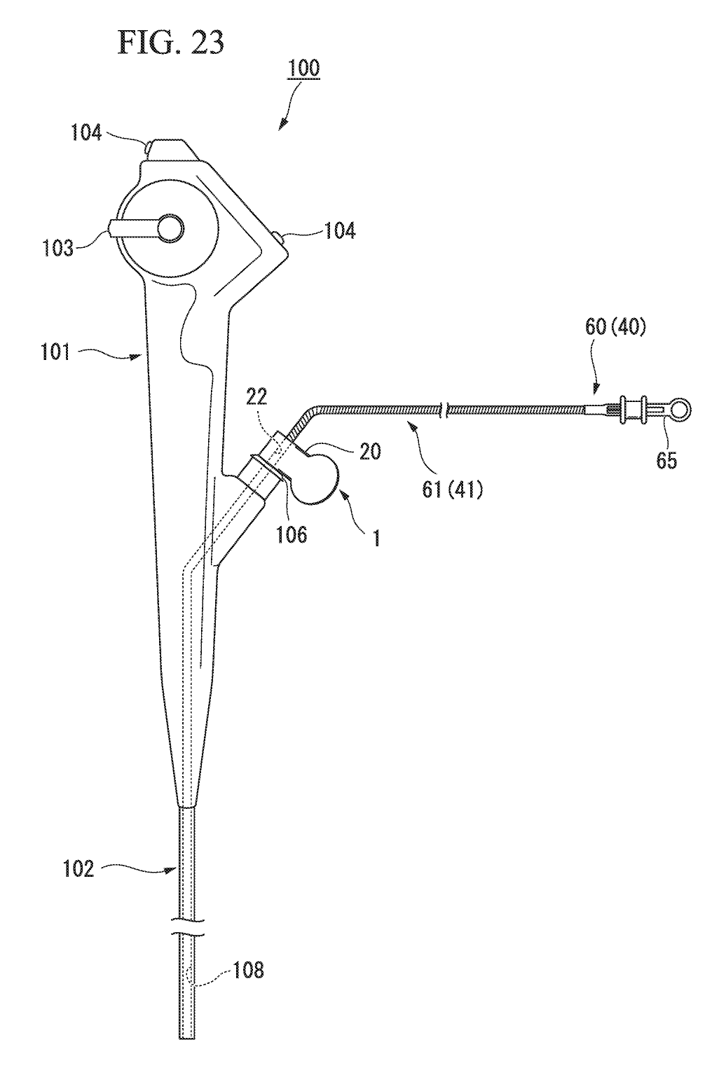

While the medical clip 1 is used with the guide sheath 30 in the description of the above-mentioned embodiment, the guide sheath 30 may not be used. FIG. 23 is a view showing a use example of the medical clip 1 according to the embodiment. As shown in FIG. 23, the forceps 60 are directly inserted through the channel 108 of the endoscope 100 without passing through the guide sheath 30. Here, the medical clip 1 is attached to the insertion section 61 of the forceps 60 and abuts the insertion port 106 of the endoscope 100. In this way, since the medical clip 1 abuts the insertion port 106 of the endoscope 100, a protrusion amount of the forceps 60 from the tip of the endoscope 100 may be kept constant.

### (Variant)

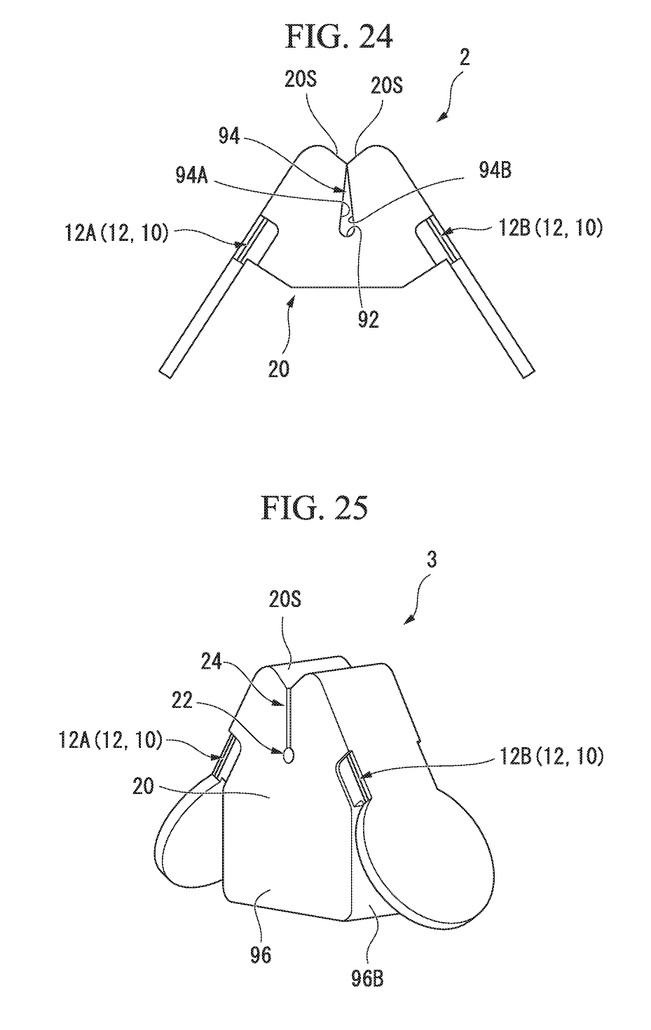

FIG. 24 is a view showing a variant of the medical clip 1 according to the embodiment. A medical clip 2 shown in FIG. 24 is distinguished from the medical clip 1 in that the edge portion 26 is not formed in the elastic member 20, and an inner surface of a through-hole 92 and an inner surface of a slit 94 are smoothly and flatly connected to each other. Specifically, a first inner surface 94A and a second inner surface 94B that are inner surfaces of the slit 94 are smoothly and flatly connected to the inner surface of the through-hole 92. In addition, the first inner surface 94A and the second inner surface 94B are formed so as to approach each other from the through-hole 92 toward the outer circumferential surface 20S. Further, in another configuration, since the medical clip 2 has the same configuration as the medical clip 1, detailed description thereof will be omitted. Even in the medical clip 2 configured as above, like the medical clip 1, attachment and removal of the medical clip 2 to/from the treatment tool 40 can be simply performed while reliably holding the treatment tool 40.

### Second Embodiment

Next, a second embodiment of the present invention will be described with reference to FIGS. 25 to 27.

FIG. 25 is a perspective view showing a medical clip 3 according to the embodiment. The medical clip 3 is distinguished from the medical clip 1 according to the first embodiment in that a restricting member 96 is further provided between the pair of knob sections 12. Further, detailed description of the components having the same configurations as those of the medical clip 1 according to the embodiment will be omitted.

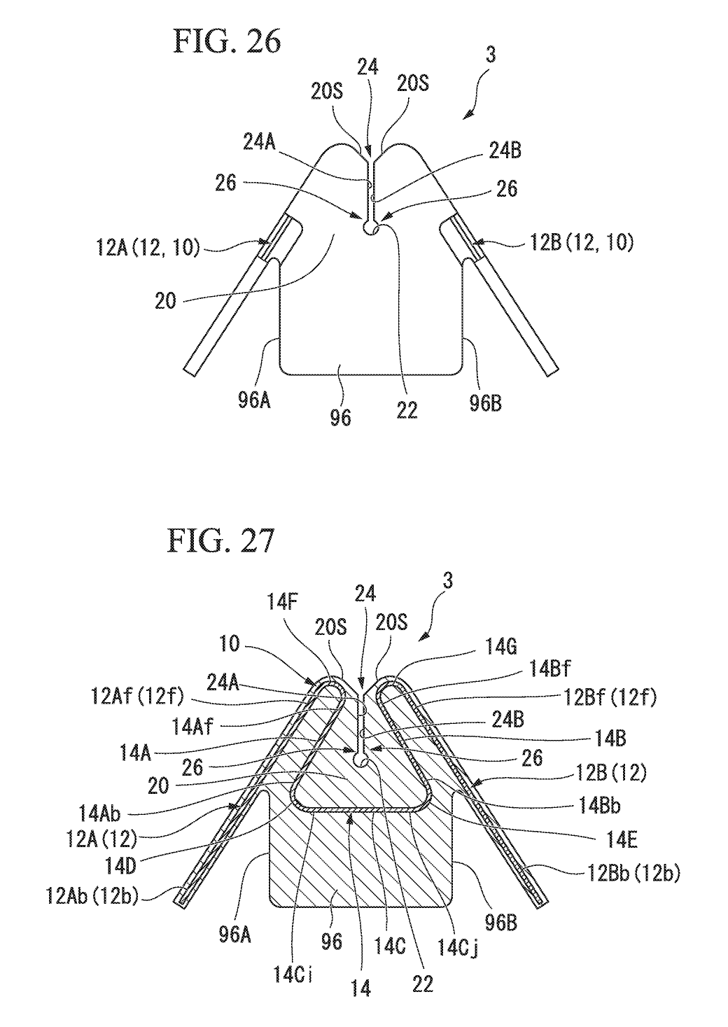

FIG. 26 is a front view showing the medical clip 3. FIG. 27 is a cross-sectional view of the medical clip 3 showing a cross section corresponding to a cross section of the medical clip 1 shown in FIG. 5. The restricting member 96 is installed between the pair of knob sections 12 and restricts a movement range in which the front end portions 12f of the pair of knob sections 12 approach and separate from each other. In the embodiment, the restricting member 96 is formed integrally with the elastic member 20 and to extend to an opposite side of the slit 24. In addition, the restricting member 96 is installed between the rear end portions 12b of the pair of knob sections 12 so as to be separated from the rear end portions 12b. A distance between a first side surface 96A of the restricting member 96 and the rear end portion 12Ab of the first knob 12A and a distance between a second side surface 96B of the restricting member 96 and the rear end portion 12Bb of the second knob 12B are set to a distance such that normal operation of the slit 24 that is opened and closed according to approaching and separation of the front end portions 12f of the pair of knob sections 12 is not hindered.

When the front end portions 12f of the pair of knob sections 12 are separated from each other, the rear end portions 12b approach the restricting member 96 while approaching each other. Then, when the front end portions 12f are separated from each other beyond a predetermined range, the rear end portions 12b abut the restricting member 96. That is, the rear end portion 12Ab of the first knob 12A abuts the first side surface 96A of the restricting member 96 via the elastic member 20 installed on the surface thereof. The rear end portion 12Bb of the second knob 12B abuts the second side surface 96B of the restricting member 96 via the elastic member 20 installed on the surface thereof. In this state, the front end portions 12f are prevented from being further separated from each other by the restricting member 96 while the rear end portions 12b are prevented from further approaching each other. Accordingly, a movement range in which the front end portions 12f approach and separate from each other is restricted by the restricting member 96. Accordingly, plastic deformation of the frame 10 or the elastic member 20 due to excessive opening of the slit 24 together with excessive separation of the front end portions 12f from each other, damage to the frame 10 itself or the elastic member 20 itself or impairment of a function of the medical clip can be prevented.

Further, while the restricting member 96 is formed integrally with the elastic member 20 in the embodiment, the elastic member 20 may be separately formed. The restricting member 96 may not have elasticity or may be formed of a member having rigidity.

In addition, the configuration of the above-mentioned restricting member 96 is only exemplary, and the configuration is not particularly limited as long as the movement range in which the front end portions 12f approach and separate from each other is restricted.

According to the medical clip 3 of the embodiment, the restricting member 96 configured to restrict the movement range in which the front end portions 12f of the pair of knob sections 12 approach and separate from each other is installed between the pair of knob sections 12. Accordingly, plastic deformation of the frame 10 or the elastic member 20 due to excessive opening the slit 24 together with excessive separation of the front end portions 12f from each other, damage to the frame 10 itself or the elastic member 20 itself, or impairment of a function of the medical clip 3 can be prevented. In addition, even in the medical clip 3 according to the embodiment, the same effect as the medical clip 1 according to the above-mentioned first embodiment is obtained.

### Third Embodiment

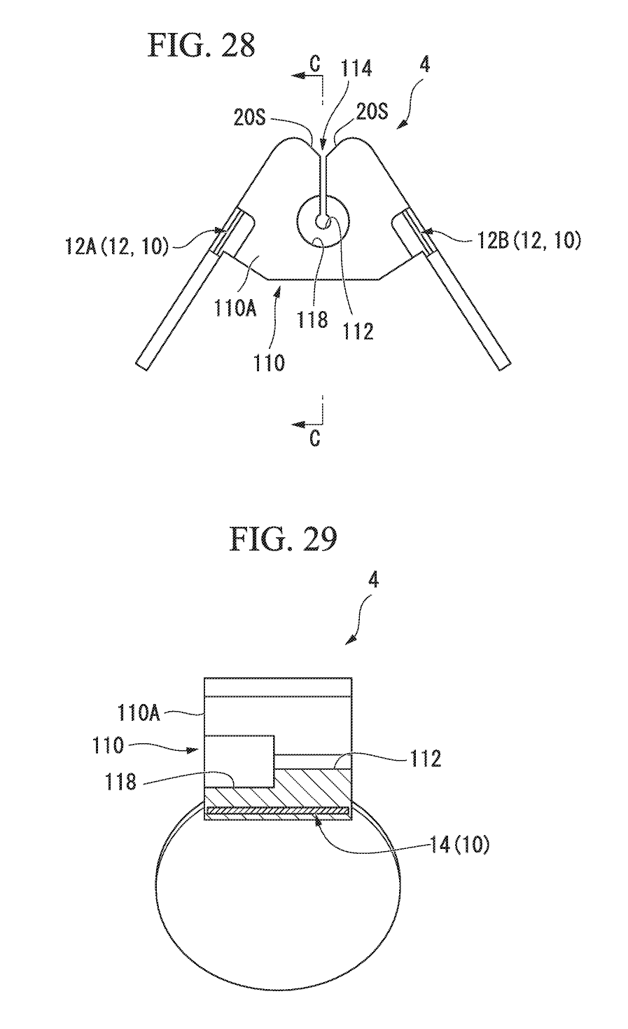

Next, a third embodiment of the present invention will be described with reference to FIGS. 28 to 30.

FIG. 28 is a front view showing a medical clip 4 according to the embodiment. The medical clip 4 is distinguished from the medical clip 1 according to the first embodiment in that an elastic member 110 further has a fitting groove 118 that can be fitted onto the base end portion 36 of the guide sheath 30. Further, detailed description of the components having the same configurations as those of the medical clip 1 according to the first embodiment will be omitted.

FIG. 29 is a cross-sectional view taken along line C-C in FIG. 28. The fitting groove 118 comes in communication with a through-hole 112 and a slit 114 and is formed in a surface of the elastic member 110 crossing the first direction and about the through-hole 112. In the embodiment, the fitting groove 118 is formed in a surface 110A that is a surface of the elastic member 110 crossing the second direction perpendicular to the first plane so as to be recessed about the through-hole 112. That is, the fitting groove 118 is installed in the vicinity of the through-hole 112 in the second direction. The fitting groove 118 has an inner diameter smaller than the outer diameter of the base end portion 36 of the guide sheath 30. Since the outer diameter of the base end portion 36 of the guide sheath 30 is larger than the outer diameter of the insertion section 41 of the treatment tool 40, the inner diameter of the fitting groove 118 is larger than the inner diameter of the through-hole 112. In addition, a dimension of the fitting groove 118 in the second direction is set to a dimension such that the base end portion 36 can be fitted into the fitting groove 118. Since the dimension of the elastic member 110 in the second direction is set to be substantially equal to the dimension of the frame 10 in the widthwise direction, the elastic member 110 of the portion in which the fitting groove 118 is formed is also surrounded and supported by the connecting section 14 of the frame 10 like the elastic member 110 of the portion in which the through-hole 112 is formed.

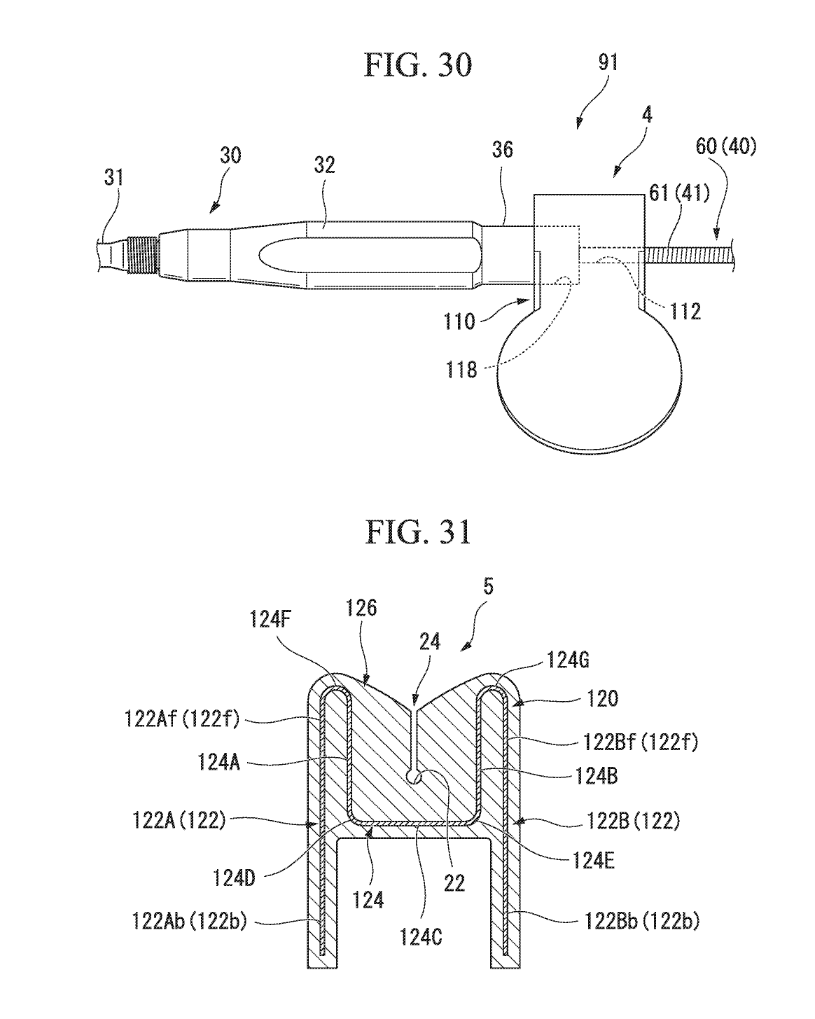

FIG. 30 is a view showing a state in which the medical clip 4 attached to the forceps 60 is fitted onto the base end portion 36 of the guide sheath 30. As an example, FIG. 30 shows a treatment tool system 91 including the medical clip 4, the guide sheath 30 and the forceps 60 (the treatment tool 40) according to the embodiment. When the fitting groove 118 is fitted onto the base end portion 36 of the guide sheath 30 in a state in which the medical clip 4 is attached to the forceps 60, the elastic member 110 is elastically deformed such that the fitting groove 118 follows the outline of the base end portion 36. Accordingly, the medical clip 4 holds and fixes the base end portion 36 by bringing the outer circumferential surface of the base end portion 36 and the inner surface of the fitting groove 118 in surface contact with each other while the elastic member 110 presses the base end portion 36 using a recovering force of returning the elastic member 110 to a shape before elastic deformation. As a result, the forceps 60 can be fixed to the guide sheath 30 via the medical clip 4, and the forceps 60 can be prevented from moving forward and rearward in the guide sheath 30. In addition, since the elastic member 110 of the portion in which the fitting groove 118 is formed is supported by the connecting section 14 of the frame 10, the medical clip 4 can more reliably hold the base end portion 36 of the guide sheath 30.

According to the medical clip 4 of the embodiment, the elastic member 110 further has the fitting groove 118 in communication with the through-hole 112 and the slit 114, formed in the surface 110A crossing the elastic member 110 in the second direction about the through-hole 112 and fittable onto the base end portion 36 of the guide sheath 30. Accordingly, since the fitting groove 118 is fitted onto the base end portion 36 of the guide sheath 30, the forceps 60 can be fixed to the guide sheath 30 via the medical clip 4. Accordingly, the forceps 60 can be prevented from moving forward and rearward in the guide sheath 30. In addition, even in the medical clip 4 according to the embodiment, the same effect as the medical clip 1 of the above-mentioned first embodiment is obtained.

### Fourth Embodiment

Next, a fourth embodiment of the present invention will be described with reference to FIG. 31.

FIG. 31 is a view showing a medical clip 5 according to the embodiment. Further, FIG. 31 shows a cross section of the medical clip 5 corresponding to the cross section of the medical clip 1 shown in FIG. 5. The medical clip 5 is distinguished from the medical clip 1 according to the first embodiment in that a pair of knob sections 122 of a frame 120 are disposed parallel to each other. Further, detailed description of the components having the same configurations as those of the medical clip 1 according to the first embodiment will be omitted.

A first knob 122A and a second knob 122B of the pair of knob sections 122 are disposed parallel to each other. Accordingly, a distance between a front end portion 122Af of the first knob 122A and a front end portion 122Bf of the second knob 122B is equal to a distance between a rear end portion 122Ab of the first knob 122A and a rear end portion 122Bb of the second knob 122B.

The first knob 122A and the second knob 122B are connected to each other by a connecting section 124. Like the medical clip 1 according to the first embodiment, a first portion 124A of the connecting section 124 is disposed substantially parallel to the first knob 122A, and connected to the first knob 122A via a third bent section 124F. In addition, a second portion 124B of the connecting section 124 is disposed substantially parallel to the second knob 122B, and connected to the second knob 122B via a fourth bent section 124G In the embodiment, since the first knob 122A and the second knob 122B are disposed parallel to each other, the first portion 124A and a third portion 124C are connected via a first bent section 124D while being disposed perpendicular to each other. Similarly, the second portion 124B and the third portion 124C are connected via a second bent section 124E while being disposed perpendicular to each other.

Like the medical clip 1 according to the first embodiment, the through-hole 22 and the slit 24 are formed in an elastic member 126 installed between front end portions 122f of the pair of knob sections 122.

Even in the medical clip 5 configured as above, like the medical clip 1 according to the first embodiment, since rear end portions 122b of the pair of knob sections 122 approach each other, the front end portions 122f can be separated from each other. Then, according to the operation of the front end portions 122f, the slit 24 can be opened. In addition, the slit 24 can be closed by returning the separated front end portions 122f to their original position (approaching each other). Accordingly, even in the medical clip 5 according to the embodiment, the same effect as the medical clip 1 according to the above-mentioned first embodiment is obtained.

### Fifth Embodiment

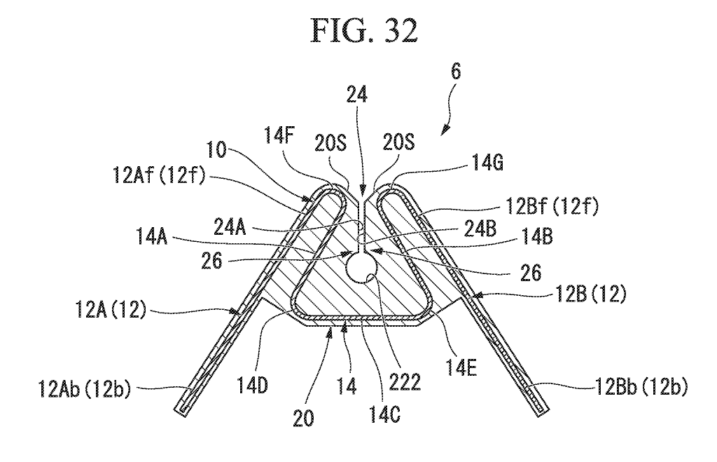

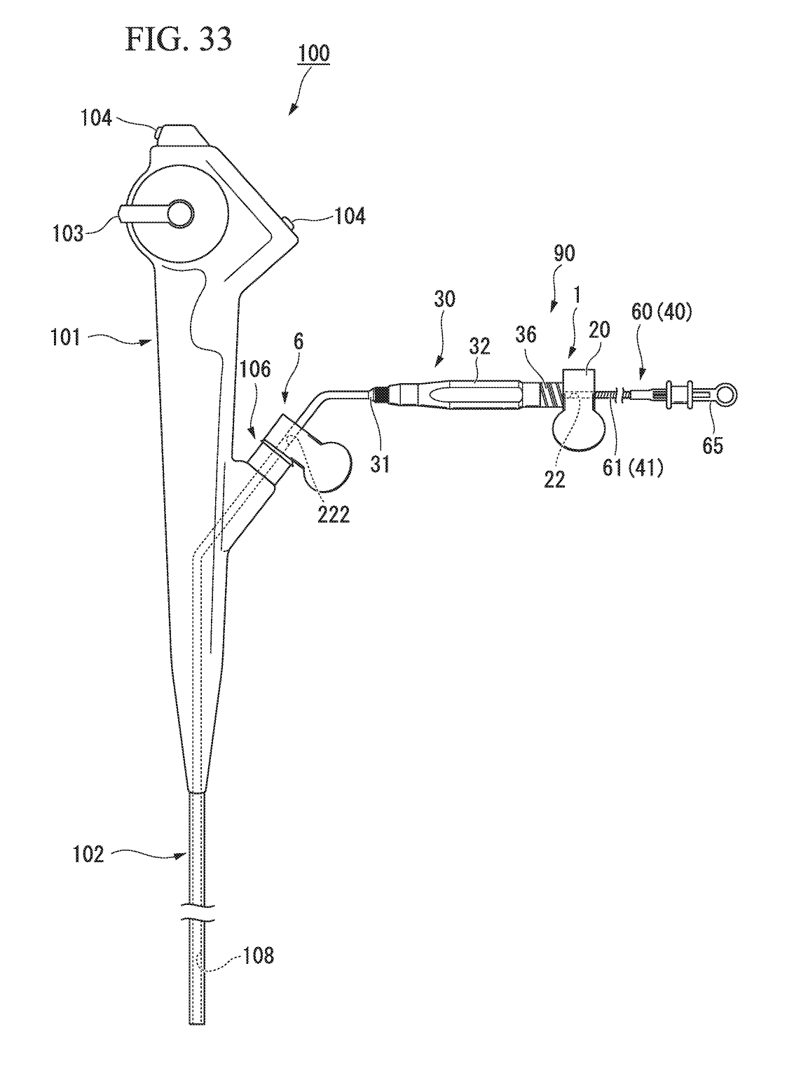

Next, a fifth embodiment of the present invention will be described with reference to FIGS. 32 and 33.

FIG. 32 is a view showing a medical clip 6 according to the embodiment. Further, FIG. 32 shows a cross section of the medical clip 6 corresponding to the cross section of the medical clip 1 shown in FIG. 5. The medical clip 6 is attached to the sheath main body 31 of the guide sheath 30 for use. The medical clip 6 is distinguished from the medical clip 1 according to the first embodiment in that a through-hole 222 is formed instead of the through-hole 22. Further, detailed description of the components having the same configurations as those of the medical clip 1 according to the first embodiment will be omitted.

The through-hole 222 is formed to pass through the elastic member 20 in a direction crossing a plane passing through the front end portions 12f and the rear end portions 12b of the pair of knob sections 12. In addition, the through-hole 222 has an inner diameter smaller than the outer diameter of the sheath main body 31 of the guide sheath 30 while being not elastically deformed. Since the through-hole 222 is configured in this way, when the sheath main body 31 of the guide sheath 30 is engaged with the through-hole 222, the elastic member 20 is elastically deformed such that the through-hole 222 follows the outline of the sheath main body 31. Accordingly, the medical clip 6 can hold the guide sheath 30.

FIG. 33 is a view showing a use example of the medical clip 6. As shown in FIG. 33, since the medical clip 6 attached to the sheath main body 31 of the guide sheath 30 abuts the insertion port 106 of the endoscope 100, a relative position of the sheath main body 31 with respect to the insertion section 102 can be fixed. That is, the protrusion amount of the sheath main body 31 from the tip of the insertion section 102 can be kept constant. In addition, for example, the medical clip 6 may be used instead of the guide sheath stopper used as the marker marked on the guide sheath 30 in Procedure Example 1 described in the first embodiment.

Hereinabove, although preferable embodiments of the present invention have been described, the present invention is not limited to the embodiments. Additions, omissions, substitutions and other modifications of configurations may be made without departing from the spirit of the present invention.

### Supplementary Note 1

A treatment method of treating a target area of a living body tissue using an endoscope having a channel, the treatment method comprising:

inserting a guide sheath through the channel and causing a tip portion of an ultrasound probe protruding from a tip of the guide sheath to approach the target area from a lumen of the living body tissue in a state in which the ultrasound probe formed to extend from a base end portion to the tip portion and configured to oscillate ultrasound waves is inserted through the guide sheath;

connecting a medical clip to the base end portion of the ultrasound probe on a base end side of the guide sheath from a direction perpendicular to a lengthwise direction of the ultrasound probe to set a protrusion amount of the tip portion of the ultrasound probe from the tip of the guide sheath toward the target area in a state in which the tip portion of the ultrasound probe approaches the target area;

drawing out the ultrasound probe from the guide sheath in a state in which the medical clip is connected to the base end portion of the ultrasound probe;

connecting a medical clip to a base end portion of a treatment tool from a direction perpendicular to the lengthwise direction of the treatment tool such that the treatment tool configured to treat the target area extends from the tip of the guide sheath by the same protrusion amount as the set protrusion amount set for the tip portion of the ultrasound probe;

inserting the guide sheath through the channel in a state in which the treatment tool is inserted through the guide sheath, and causing the tip of the treatment tool to approach the target area such that the treatment tool extends by the set protrusion amount from the tip of the guide sheath by the medical clip; and

treating the target area using the tip of the treatment section.

### Supplementary Note 2

The treatment method according to Supplementary Note 1, wherein the tip of the guide sheath approaches the target area together with the tip portion of the ultrasound probe.

### Supplementary Note 3

The treatment method according to Supplementary Note 1, wherein the tip of the guide sheath is stopped at a position closer to the tip of the endoscope than the target area when the tip portion of the ultrasound probe approaches the target area.

### Supplementary Note 4

A treatment method of treating a target area of a living body tissue using an endoscope having a channel, the treatment method comprising:

inserting an ultrasound probe formed to extend from a base end portion to a tip portion and configured to oscillate ultrasound waves through the channel, and causing the tip portion of the ultrasound probe protruding from a tip of the endoscope to approach the target area from a lumen of the living body tissue;

connecting a medical clip to the base end portion of the ultrasound probe on a base end side of the channel from a direction perpendicular to a lengthwise direction of the ultrasound probe to set a protrusion amount of the tip portion of the ultrasound probe from the tip of the endoscope toward the target area in a state in which the tip portion of the ultrasound probe approaches the target area;

drawing out the ultrasound probe from the channel in a state in which the medical clip is connected to the base end portion of the ultrasound probe;

connecting a medical clip to a base end portion of a treatment tool from a direction perpendicular to a lengthwise direction of the treatment tool such that the treatment tool configured to treat the target area extends from the tip of the endoscope by the same protrusion amount as the set protrusion amount of the tip portion of the ultrasound probe;

inserting the treatment tool through the channel, and causing the tip of the treatment tool to approach the target area such that the treatment tool extends by the set protrusion amount from the tip of the endoscope by the medical clip; and

treating the target area using the tip of the treatment section.

According to the above-mentioned medical clip and treatment tool system, attachment and removal to/from a treatment tool can be simply performed while reliably holding the treatment tool.

What is claimed is:

1. A medical clip attached to a treatment tool inserted and used in an endoscope, the medical clip comprising:
a pair of knob sections formed to extend from front end portions to rear end portions and constituted by a first knob and a second knob, the first knob and the second knob being formed so as to be separated from each other, the first knob being disposed parallel to a first portion having a flat plate shape, the second knob being disposed parallel to a second portion having a flat plate shape;
a connecting section including the first portion, the second portion, a first bent section, a second bent section, a third bent section, and a fourth bent section, wherein the first portion is connected to the second portion via the first bent section and the second bent section, the first knob is connected to the first portion via the third bent section, and the second knob is connected to the second portion via the fourth bent section; and
an elastic member installed between the front end portion of the first knob and the front end portion of the second knob and elastically deformable,
wherein a through-hole is formed in the elastic member to pass through the elastic member in a direction crossing an opening and closing direction of the first knob and the second knob, and
the elastic member has a slit formed to extend from the through-hole to an outer circumferential surface of the elastic member toward the front end portions of the pair of knob sections and having a gap in a state in which the front end portion of the first knob and the front end portion of the second knob approach and separate from each other.

2. The medical clip according to claim 1, wherein the elastic member further has an edge portion that forms a dihedral angle between an inner surface of the through-hole and an inner surface of the slit.

3. The medical clip according to claim 1, further comprising:
a restricting member installed between the pair of knob sections and configured to restrict a movement range in which the front end portions of the pair of knob sections approach and separate from each other.

4. The medical clip according to claim 1, wherein a fitting groove is formed in communication with the through-hole and the slit and is formed in a surface of the elastic member crossing the direction about the through-hole,
the fitting groove is configured to be able to be fitted onto a base end portion of the guide sheath in a state in which the treatment tool is inserted into a guide sheath, and
the fitting groove has an inner diameter smaller than an outer diameter of the base end portion of the guide sheath and larger than the inner diameter of the through-hole and is formed so as to be recessed from a surface of the elastic member crossing the direction.

5. A treatment tool system comprising:
a guide sheath formed to extend from a tip to a base end and able to be endoscopically inserted into a living body;
a treatment tool having an insertion section that is able to be inserted into the guide sheath and a treatment section installed on a tip of the insertion section and configured to perform observation or treatment of the living body; and
a medical clip detachably attached to the insertion section,
wherein the medical clip comprises:
a pair of knob sections formed to extend from front end portions to rear end portions and constituted by a first knob and a second knob, the first knob and the second knob being formed so as to be separated from each other, the first knob being disposed parallel to a first portion having a flat plate shape, the second knob being disposed parallel to a second portion having a flat plate shape;
a connecting section including the first portion, the second portion, a first bent section, a second bent section, a third bent section, and a fourth bent section, wherein the first portion is connected to the second portion via the first bent section and the second bent section, the first knob is connected to the first portion via the third bent section, and the second knob is connected to the second portion via the fourth bent section; and
an elastic member installed between the front end portion of the first knob and the front end portion of the second knob and elastically deformable,
wherein a through-hole is formed in the elastic member to pass through the elastic member in a direction crossing an opening and closing direction of the first knob and the second knob, and
the elastic member has a slit formed to extend from the through-hole to an outer circumferential surface of the elastic member toward the front end portions of the pair of knob sections and having a gap in a state in which the front end portion of the first knob and the front end portion of the second knob approach and separate from each other.

6. The treatment tool system according to claim 5, wherein the elastic member further has an edge portion that forms a dihedral angle between an inner surface of the through-hole and an inner surface of the slit.

7. The treatment tool system according to claim 5, wherein the medical clip further comprises a restricting member installed between the pair of knob sections and configured to restrict a movement range in which the front end portions of the pair of knob sections approach and separate from each other.

8. The treatment tool system according to claim 5, wherein the treatment tool is any one of an ultrasound probe configured to observe a target tissue in the living body through ultrasound, forceps configured to grasp the target tissue, a brush configured to exfoliate a surface layer of the target tissue, and a guiding device configured to guide the guide sheath in the living body.

9. A method of applying a medical clip, the method comprising:
(1) inserting an ultrasound probe inserted through a guide sheath into a bronchiole;
(2) causing an ultrasound probe to extend from a tip of the guide sheath and specifying a target area;
(3) marking a mark on a base end of the ultrasound probe and defining a length of the ultrasound probe inserted into the guide sheath after defining the target area;
(4) inserting and detaching the ultrasound probe into/from the guide sheath;
(5) positioning the mark at the same position as a length of the ultrasound probe from the position at which the mark of the base end of the ultrasound probe is marked to a tip of the ultrasound probe by pinching a clip at a base end position of an insertion section of a treatment section from an outer side surface of the insertion section of the treatment section toward a major axis of the insertion section;
(6) inserting the treatment section through the guide sheath and inserting a tip of the treatment section toward a target area, and causing the clip to butt against a base end surface of the guide sheath; and
(7) treating a living body tissue in the target area using the treatment section in a state in which the clip butts against a base end of the guide sheath.

10. The method of applying the medical clip according to claim 9, wherein the clip comprises a pair of knob sections formed to extend from front end portions to rear end portions and constituted by a first knob and a second knob, the first knob and the second knob being formed so as to be separated from each other,
the pair of knob sections are disposed parallel to a first portion having a flat pate shape and a second portion having a flat plate shape,
the first portion and the second portion have a connecting section connected by a first bent section and a second bent section,
the pair of knob sections are connected by a third bent section and a fourth bent section to connect the first portion and the second portion,
an elastic member installed between the front end portion of the first knob and the front end portion of the second knob and elastically deformable is provided,
a through-hole is formed in the elastic member to pass through the elastic member in a direction crossing a plane passing through the front end portion and the rear end portion of the pair of knob sections,
the elastic member has a slit having a gap in communication with the through-hole throughout the length of the through-hole and extending to an outer circumferential surface of the elastic member in a state in which the front end portion of the first knob and the front end portion of the second knob approach each other, and
through the clip, the insertion section of the treatment section is inserted from the slit toward the through-hole and further positioned.

11. The method of applying the medical clip according to claim 10, wherein the elastic member includes an edge portion that forms a dihedral angle between an inner surface of the through-hole and an inner surface of the slit, and
the clip is movable in a lengthwise direction of the insertion section of the treatment section and able to be detachably positioned from the insertion section by the edge portion.

12. The method of applying the medical clip according to claim 9, wherein a movement range in which the front end portions of the pair of knob sections approach and separate from each other is restricted when a user opens and closes the pair of knob sections using a restricting member installed between the pair of knob sections and installed at rear end sides of the pair of knob sections.
